(19) United States
(12) Patent Application Publication
(10) Pub. No.: US 2024/0277468 A1
(43) Pub. Date: Aug. 22, 2024

(54) HEART VALVE REPAIR PROSTHESES, DELIVERY DEVICES AND METHODS

(71) Applicant: Cedars-Sinai Medical Center, Los Angeles, CA (US)

(72) Inventors: Robert James Siegel, Beverly Hills, CA (US); Lawrence E. Ong, Beverly Hills, CA (US); Miles Alexander, Fremont, CA (US)

(21) Appl. No.: 18/568,995

(22) PCT Filed: Jun. 13, 2022
(86) PCT No.: PCT/US2022/033315

§ 371 Date: Dec. 11, 2023

Related U.S. Application Data
(60) Provisional application No. 63/210,898, filed on Jun. 15, 2021.

(51) Int. Cl. A61F 2/24

(52) U.S. Cl. CPC A61F 2/2418; A61F 2/2433; A61F 2/2436

(57) ABSTRACT

A heart valve prosthesis including separable ventricular and members is provided. The ventricular member has a first hub that has a connection feature. Each of at least three arms are coupled at a first end to the first hub and have a second end opposite the first end. The arms are preloaded toward a location of heart valve leaflet when the ventricular member is implanted. The atrial member has a second hub coupled to a frame array and has a connection feature configured to couple to the connection feature of the first hub for securing the atrial and ventricular members together. The frame array is preloaded toward a location of the heart valve leaflets when the atrial member is implanted. Concavities are formed at the periphery of the frame array to receive the arms of the ventricular member in an engaged configuration. Valve tissue may be trapped between the arms and the concavities.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 C.F.R. § 1.57. For example, the present application claims priority to U.S. Provisional Patent Application No. 63/210,898, filed Jun. 15, 2021, which is incorporated herein by reference in its entirety.

### BACKGROUND OF THE INVENTION

### Field of the Invention

This application is directed to heart valve prostheses configured to treat valve regurgitation, preferably without surgery.

### Description of the Related Art

Heart valve regurgitation is a condition that arises from various causes in which blood flows backward through the valve when the valve should have prevented such flow. Regurgitation can occur in several valves of the heart, including the mitral valve, the aortic, valve, the pulmonary valve and the tricuspid valve.

The tricuspid valve separates the right lower heart chamber (the right ventricle) from the right upper heart chamber (right atrium). Tricuspid regurgitation is a disorder in which this valve does not close tight enough. This problem causes blood to flow backward into the right upper heart chamber (atrium) when the right lower heart chamber (ventricle) contracts. Tricuspid regurgitation is leakage of blood backwards through the tricuspid valve each time the right ventricle contracts. Tricuspid regurgitation usually results from an enlarged lower heart chamber (called the ventricle) or from any other condition that constrains the blood flow from the right ventricle to the lungs. Sometimes long-standing disorders, such as emphysema or pulmonary stenosis can cause problems that affect the tricuspid valve which is “upstream” from the lungs. To compensate, the right ventricle enlarges so that it can pump harder, which sometimes causes the tricuspid opening to become stretched out and floppy.

Valve repair is the most common surgical treatment for tricuspid valve disease. This procedure can be done alone or in combination with treatments for other heart problems. Tricuspid valve repair using an annuloplasty ring is a common surgical approach for tricuspid regurgitation and may be performed for primary tricuspid disease or for combined cases with other valve surgery (mitral, aortic). Traditional tricuspid valve repair is an open-heart procedure performed through a 6-8 inch incision through the breastbone (sternum).

While transcatheter systems for tricuspid repair have been explored, such systems provide suboptimal acute results, are complex to use, lacks durability, and poses a too high risk of valve thrombosis unless patients are anticoagulated.

### SUMMARY OF THE INVENTION

For these reasons, there exists a need for minimally invasive methods of tricuspid valve repair. The present disclosure is directed to valve fixation devices that can be delivered endovascularly.

In one embodiment, a heart valve prosthesis is provided that includes a ventricular member and an atrial member. The ventricular member is configured to be advanced into a ventricle of a heart. The ventricular member has a hub (sometimes referred to as a first hub) that includes one or more slots and an array of arms. The first hub can be configured to be disposed adjacent to the line of coaptation of a first heart leaflet and a second heart leaflet. The first hub can be positioned on the ventricular side of the line of coaptation, within the line of coaptation and/or into the atrial side of the line of coaptation. Each arm of the array of arms can have a first end connected to the first hub and a second end opposite the first end. The second end of one or more arms (e.g., of each arm) can be deflected towards the first hub in a free state. The second ends of adjacent arms are not connected to each other. For example, the second ends of adjacent arms are not connected to each other in the circumferential direction in some embodiments. The arms are not connected to each other along their length between the first end and the second end in some embodiments. One or more of the second ends of the arms can be configured to be placed into direct contact with the first heart leaflet. One or more of the second ends of the arms can be configured to be placed into direct contact with the second heart leaflet. The atrial member is configured to be advanced into an atrium adjacent to the line of coaptation. The atrial member can be positioned across (e.g., spanning the gap between) the line of coaptation of the first and second heart leaflets. The atrial member can be positioned across (e.g., on an opposite side of) the line of coaptation of the first and second heart leaflets from the arms of the ventricular member. The atrial member has a second hub that has one or more, e.g., a plurality of tangs, and an array of petals. The tangs can be configured for locking into the slots of the first hub to engage the second hub with the first hub when the atrial member and the ventricular member are assembled. One or more petal, e.g., each petal, of the array of petals has a base end connected to the second hub and an outer end opposite the base end. The outer end of one or more petal, e.g., each petal, is deflected away from the second hub. One or more adjacent petals, e.g., each adjacent petal, is bounded by a shared inner strut and a separate outer strut forming a concavity at the junction of the shared inner strut and the separate outer strut. The atrial member is separate from and moveable relative to the ventricular member prior to the first hub and the second hub being engaged. In an engaged configuration the arms of the ventricular member are pressed into the concavity of the atrial member.

A ventricular member of a heart valve prosthesis can be provided. A ventricular member or clamp member can have a plurality of arms disposed around a central member. The arms can collectively span two or three heart leaflets. Each arm of the plurality of arms can have a free end deflected towards the central member. The second ends of adjacent arms are not connected to each other. For example, the second ends of adjacent arms are not connected to each other in the circumferential direction in some embodiments. The arms are not connected to each other along their length between the first end and the second end in some embodiments. One or more of the second ends of the arms can be configured to be placed into direct contact with the first heart leaflet. One or more of the second ends of the arms can be configured to be placed into direct contact with the second heart leaflet.

The central member of the ventricular member or ventricular clamp can include a hub. The hub can have slots or tangs for engagement with another valve leaflet capture member, e.g., an atrial clamp.

An atrial member or atrial clamp of a heart valve prosthesis can be provided. The atrial member can be positioned across, e.g., on an opposite side of, the first and second heart leaflets from a mating ventricular member or clamp. The atrial member can have a central member and an array of petals. One or more petal, e.g., each petal, of the array of petals is connected to the central member and has an outer end opposite the central member. The outer end of one or more petal, e.g., each petal, is deflected away from the central member. The petals can include an array of frame members that are symmetrical and symmetrically disposed around a center of the central member of the atrial member. Non-symmetrical (or asymmetrical) frames or symmetrical frames arranged in a non-symmetrical (or asymmetrical) manner can be provided in some embodiments.

One or more adjacent petals, e.g., each adjacent petal, can be bounded by a radial strut adjacent to the central member. The radial strut can be or include a shared inner strut. The petals can be bounded by separate outer struts. A concavity at the junction of the shared inner strut and the separate outer strut.

If provided together in a heart valve prosthesis, the atrial member can be separate from and moveable relative to the ventricular member prior to implantation. The central members of the ventricular and atrial members can be engaged following movement of the ventricular and atrial members together. In an engaged configuration, arms of the ventricular member can be pressed against the atrial member, e.g, into a concavity formed between adjacent petals of the atrial member, such that a valve leaflet is compressed therebetween. In an engaged configuration, the atrial member can deflect one or more arms such that a valve leaflet is compressed therebetween. In an engaged configuration, the petals of the atrial member and the arms of the ventricular member can both deflect such that a valve leaflet is compressed therebetween.

A portion of the ventricular member can be positioned across the line of coaptation of the first and second heart leaflets (e.g., spanning the gap between the leaflets) when the ventricular member is engaged with the atrial member. The atrial member can include slots or tangs to engage the ventricular member. If the atrial member includes tangs, the tangs can be configured for locking into slots disposed on the central member of the ventricular member when the atrial member and the ventricular member are assembled. If the atrial member includes slot, the slots can be configured for locking into tangs projecting from the central member of the ventricular member when the atrial member and the ventricular member are assembled.

In another embodiment, a heart valve prosthesis is provided that includes a ventricular member and an atrial member. The atrial member is separate from and moveable relative to the ventricular member, e.g., prior to assembly. The atrial member and the ventricular member are configured for assembly in the heart in some embodiments. For example, a connection feature of a first hub of the ventricular member and a connection feature of a second hub of the atrial member can be engaged inside the heart. The ventricular member can be configured to be advanced into a ventricle of a heart. The ventricular member can have a first hub having a slot and at least three arms. The first hub can be configured to be disposed adjacent to the line of coaptation of a first heart leaflet and a second heart leaflet. Each arm can have a first end connected to the first hub and a second end opposite the first end, the second ends of adjacent arms are not connected to each other. One or more of the second ends are configured to be placed into direct contact with the first heart leaflet and one or more of the second ends are configured to be placed into direct contact with the second heart leaflet. The atrial member can be configured to be advanced at least partially into an atrium across the line of coaptation of the first and second heart leaflets. The atrial member has a second hub that has a tang. The atrial member has a frame array. The tang of the second hub is configured for locking into the slot of the first hub to engage the second hub with the first hub when the atrial member and the ventricular member are assembled. The frame array includes a plurality of frames. The frames have a base portion connected to the second hub and an outer portion opposite the base portion. Each frame of the frame array has two shared struts and two dedicated strut portions. The two shared struts are connected to the second hub at a first end and forming the base portion. The two dedicated strut portions attached at a second end of each of the shared struts. One or more frame, e.g., each frame, encloses an approximately quadrilateral shaped area. Adjacent frames are connected by shared struts. Adjacent frames form a concavity there between. In an engaged configuration, the arms of the ventricular member are pressed into the concavities of the atrial member.

In another embodiment, a heart valve prosthesis includes a ventricular member and an atrial member. The atrial member can be separate from and moveable relative to the ventricular member, e.g., prior to engagement. During engagement of the ventricular member to the atrial member, a centering feature of the atrial member guides an arm of the ventricular member into a predefined position and/or orientation as connection feature(s) (e.g., tangs) of one of the ventricular and atrial members (e.g., a hub of the atrial member) engage(s) connection feature(s) of the other of the ventricular and atrial members (e.g., slots of a first hub of the ventricular member). The ventricular member is configured to be advanced into a ventricle of a heart. The ventricular member includes a first hub and at least three arms. The first hub has a slot. The first hub is configured to be disposed adjacent to the line of coaptation of a first heart leaflet and a second heart leaflet. Each arm of the at least three arms has a first end connected to the first hub and a second end opposite the first end. One or more of the second ends of the arms is configured to be placed into direct contact with the first heart leaflet and one or more of the second ends configured to be placed into direct contact with the second heart leaflet. The atrial member is configured to be advanced into an atrium and to be positioned across the line of coaptation. The atrial member includes a second hub and at least one centering feature. The second hub has a connection feature (e.g., a tang) for locking into a connection feature (e.g., the slot of the first hub) when the atrial member and the ventricular member are assembled. The at least one centering feature is configured for centering at least one arm of the ventricular member. During an engagement of the ventricular member to the atrial member, the centering feature of the atrial member guides the arm of the ventricular member into a predefined position and/or orientation as the connection feature (e.g., tangs) of the second hub engage the connection features (e.g., slots) of the first hub.

In variations of the foregoing, a span of one or more of the arms adjacent to the second ends can be configured to be placed into direct contact with the first heart leaflet. A span of one or more of the arms adjacent to the second ends can be configured to be placed into direct contact with the second heart leaflet. A span of one or more of the arms extending from the second ends can be configured to be placed into direct contact with the first heart leaflet. A span of one or more of the arms extending from the second ends can be configured to be placed into direct contact with the second heart leaflet. An entirety of a span of one or more of the arms can be configured to be placed into direct contact with the first heart leaflet. An entirety of a span of one or more of the arms can be configured to be placed into direct contact with the second heart leaflet.

Further embodiments of the foregoing can provide the first hub of the ventricular member and the second hub of the atrial member with other connection features. For example, the first hub of the ventricular member can be provided with one or more tangs, e.g., a plurality of tangs, and the second hub of the atrial member can have one or more slots, e.g. a plurality of slots. The tangs of the first hub can be configured for locking into the slots of the second hub to engage the first hub with the second hub when the ventricular member and the atrial member are assembled. In other embodiments, the first hub of the ventricular member can be configured with one or more tangs, e.g., a plurality of tangs, and with one or more slots, e.g., a plurality of slots. The second hub of the atrial member can be configured with one or more tangs, e.g., a plurality of tangs, and with one or more slots, e.g., a plurality of slots. Slots and tangs can alternate about the periphery of the first hub. Slots and tangs can alternate about the periphery of the second hub. Alternate around the hubs in this context can include providing a first connection feature followed by a second connection feature of a different type spaced apart, e.g., circumferentially spaced apart from the first connection feature.

In another embodiment, a system for delivering a heart valve prosthesis is provided. The system can include a delivery device and a prosthesis. The delivery device can include a delivery handle, a plurality of removable lockouts, a sheath assembly, and a guide handle. The delivery handle can include a housing and a plurality of sliders disposed in the housing. The plurality of removable lockouts can be configured to prevent motion of the sliders in a first state and to allow a range of motion in a second state. The sheath assembly can include an outer sheath, an inner sheath disposed within the outer sheath, the outer sheath moveable relative to the inner sheath. The guide handle can be configured to retract the outer sheath. The prosthesis can include a ventricular member and an atrial member. The ventricular member can include at least three arms, each arm having a first end coupled to a hub. The atrial member can include a hub configured to couple with the hub of the ventricular member and at least one centering featuring comprising a concavity for centering at least one of the at least three arms of the ventricular member. The delivery handle can be configured to control movement and positioning of the prosthesis within a heart by movement of the sliders and lockouts.

In variations of the foregoing, the system can include one or more of the following features. The system can include a nosecone coupled to a distal end of the outer sheath. The nosecone can include a plurality of slits configured to open the nosecone to allow the prosthesis to exit the outer sheath. The nosecone can comprise a flexible tip configured to expand to allow the prosthesis to exit the outer sheath. The nosecone can surround the outer sheath and be configured to be retracted to allow the prosthesis to exit the outer sheath. The system can include an inflatable balloon disposed within the outer sheath, the inflatable balloon configured to be inflated to prevent the prosthesis from exiting the outer sheath and configured to be deflated to allow the prosthesis to exit the outer sheath. The system can include a source of fluid coupled with the housing and configured to supply a fluid to a space disposed between an inner surface of the outer sheath and an outer surface of the inner sheath. The system can include a source of fluid coupled with the housing and configured to supply a fluid to a plurality of spaces disposed between an inner surface of the outer sheath and an outer surface of the inner sheath. The system can include a plurality of fluid sources coupled with a plurality of spaces disposed between an inner surface of the outer sheath and an outer surface of the inner sheath, one of the fluid sources of the plurality of fluid sources supplying fluid to each one of the spaces between the inner surface of the outer heath and the outer surface of the inner sheath.

In another embodiment, a method for performing a procedure in a heat is provided. The method can include advancing a delivery catheter to the heart, passing a ventricular member through the delivery catheter and into a ventricle of the heart, the ventricular member comprising at least three arms, each arm having a first end coupled to a hub, passing an atrial member through the delivery catheter and into an atrium of the heart, the atrial member comprising at least one centering feature comprising a concavity for centering at least one of the at least three arms of the ventricular member, aligning the ventricular member and the atrial member such that tissue is compressed between the ventricular member and the atrial member, inserting the hub of the ventricular member to a hub of the atrial member, and securing the ventricular member to the atrial member.

In variations of the foregoing, the system can include one or more of the following features. The delivery catheter can be coupled to a delivery handle, the delivery handle comprising a plurality of sliders disposed in a housing and a plurality of removable lockouts configured to prevent motion of the sliders in a first state and to allow a range of motion in a second state. The sliders and lockouts can be configured to pass the ventricular member and the atrial member through the delivery catheter. The tissue can be the tricuspid valve. The method can include supplying a fluid to a space disposed within the delivery catheter via a source of fluid coupled to a delivery handle coupled to the delivery catheter. The method can include supplying a fluid to a plurality of spaces disposed within the delivery catheter. The method can include opening a plurality of slits in a nosecone coupled to a distal end of the delivery catheter, wherein opening the slits allows the ventricular member and the atrial member to pass through the distal end of the delivery catheter.

In another embodiment, a method for performing a procedure in a heat is provided. The method can include passing a ventricular member through the delivery catheter and into a right ventricle of the heart, the ventricular member comprising at least three arms, each arm having a first end coupled to a hub, positioning the ventricular member against a first side of a leaflet of a tricuspid valve, passing an atrial member through the delivery catheter and into a right atrium of the heart, the atrial member comprising at least one centering feature comprising a concavity for centering at least one of the at least three arms of the ventricular member, positioning the atrial member against a second side of the leaflet of the tricuspid valve, aligning the ventricular member and the atrial member such that leaflet of the tricuspid valve is compressed between the ventricular member and the atrial member, inserting the hub of the ventricular member to a hub of the atrial member, and securing the ventricular member to the atrial member.

In variations of the foregoing, the system can include one or more of the following features. The delivery catheter can be coupled to a delivery handle, the delivery handle comprising a plurality of sliders disposed in a housing and a plurality of removable lockouts configured to prevent motion of the sliders in a first state and to allow a range of motion in a second state. The sliders and lockouts can be configured to pass the ventricular member and the atrial member through the delivery catheter. The method can include supplying a fluid to a space disposed within the delivery catheter via a source of fluid coupled to a delivery handle coupled to the delivery catheter. The source of fluid can be configured to supply a fluid to a plurality of spaces disposed within the delivery catheter. The method can include opening a plurality of slits in a nosecone coupled to a distal end of the delivery catheter, wherein opening the slits allows the ventricular member and the atrial member to pass through the distal end of the delivery catheter. The delivery catheter can be advanced through a femoral vein. The delivery catheter can be advanced through a jugular vein.

### BRIEF DESCRIPTION OF THE DRAWINGS

A more complete appreciation of the subject matter of this application and the various advantages thereof can be realized by reference to the following detailed description, in which reference is made to the accompanying drawings in which:

FIG. 1 is a perspective view of a heart valve repair device coupled with a catheter-based delivery system;

FIG. 1A depicts a cross-sectional view of a heart in normal diastole;

FIG. 1B depicts a cross-sectional view of a heart in normal systole;

FIG. 1C is a top view of a tricuspid valve of a heart in systole, the valve in need of repair due to poor to no coaptation along the edges of the leaflets;

FIG. 1D is a top view of the tricuspid valve of FIG. 1C illustrating a non-limiting selection of locations for placement of devices across the valve leaflets to improve the diseased condition of the valve;

FIG. 2 is a perspective view of a ventricular member of the heart valve prosthesis of FIG. 1 in a collapsed state;

FIG. 3 is a perspective view of the ventricular member shown in FIG. 2 in an expanded state;

FIG. 3A is a schematic drawing illustrating angulation of one arm and a hub of the ventricular member of FIG. 2 in an expanded state;

FIG. 4 is a perspective view of an atrial member of the heart valve prosthesis of FIG. 1 in a collapsed state;

FIG. 5 is a perspective view of the atrial member shown in FIG. 4 in an expanded state;

FIG. 5A is a schematic drawing illustrating angulation of one frame and a hub of the atrial member of FIG. 5 in an expanded state;

FIGS. 5B-SC shows additional embodiments of atrial members;

FIG. 6A shows a delivery system in a first delivery state;

FIG. 6B shows a delivery system of FIG. 6A in a second delivery state;

FIG. 6C shows a delivery system of FIG. 6A in a third delivery state;

FIG. 6D shows a delivery system of FIG. 6A in a fourth delivery state;

FIG. 6E shows a delivery system of FIG. 6A in a fifth delivery state;

FIG. 6F shows a delivery system of FIG. 6A in a sixth delivery state;

FIG. 6G shows an embodiment of the delivery system of FIG. 6A including a delivery handle comprising fluid sources:

FIG. 7A show a heart valve prosthesis secured in a heart, the prosthesis shown from the atrial chamber;

FIG. 7B show a ventricular chamber view of the heart valve prosthesis of FIG. 7A implanted in a heart;

FIG. 7C is a view similar to FIG. 7A showing a heart valve prosthesis secured to heart valve tissue, the prosthesis shown from the atrial member side of the heart valve prosthesis;

FIG. 7D is a view similar to FIG. 7B showing a heart valve prosthesis secured to heart valve tissue, the prosthesis shown from the ventricular member side of the heart valve prosthesis;

FIGS. 8A and 8B show another embodiment of a heart valve prosthesis in which a plug function is provided by a resealable valve is provided in a hub of a ventricular member to control flow of blood through the heart valve prosthesis;

FIGS. 9A and 9B show another embodiment of a heart valve prosthesis in which a plug function is provided by a collapsible member is provided in a hub of a ventricular member to control flow of blood through the heart valve prosthesis; and

FIGS. 10A-10E show embodiments of various nosecones to be provided at or near the distal tip of the delivery system of FIG. 6A.

More detailed descriptions of various embodiments of valve repair devices and methods useful to treat patients suffering from valve regurgitation are set forth below.

### DETAILED DESCRIPTION OF THE PREFERRED EMBODIMENT

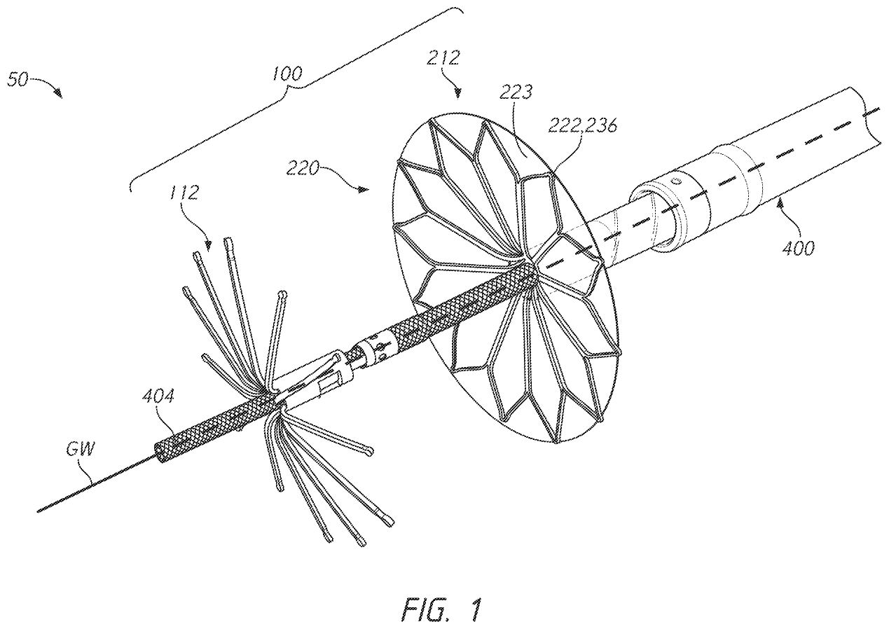

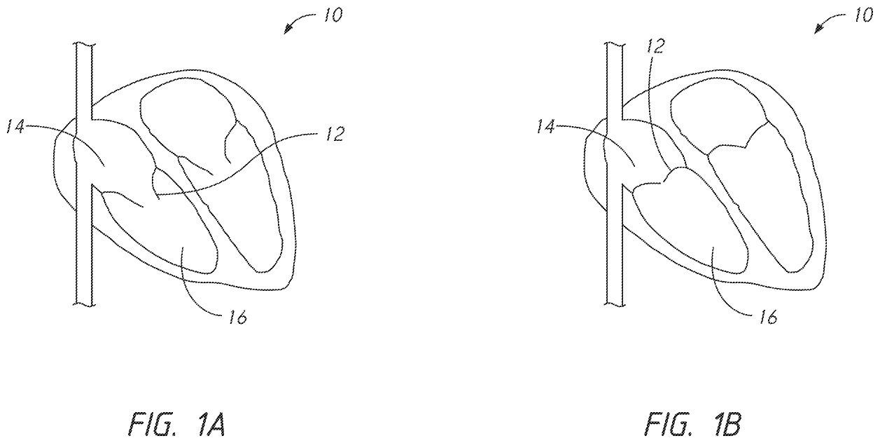

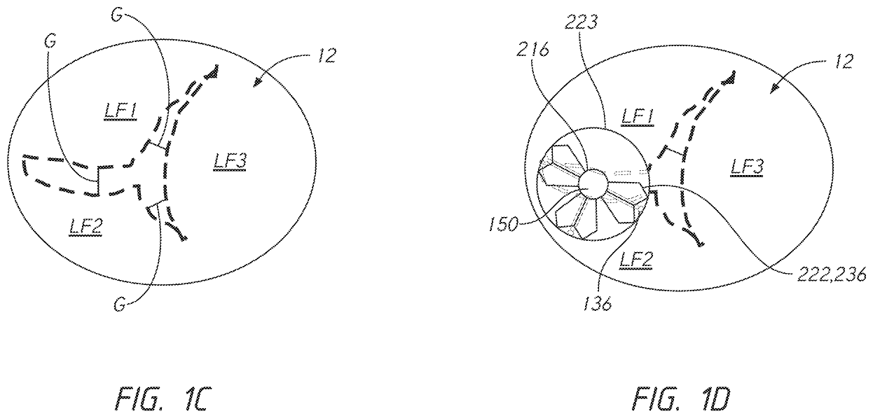

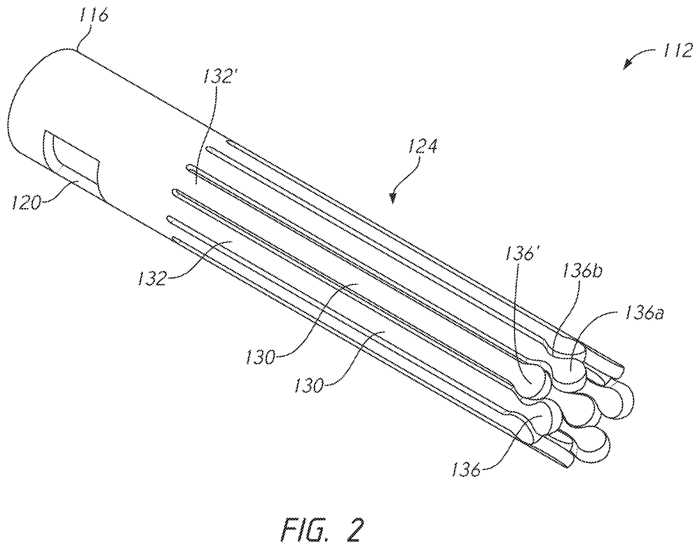

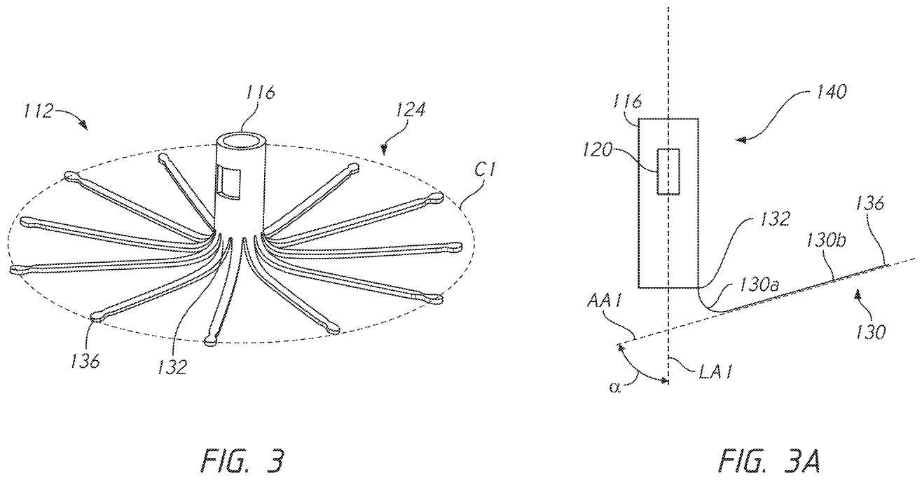

This application is directed to heart valve repair prostheses, delivery devices and methods for delivering and implanting the same. FIG. 1 illustrates one embodiment of a heart valve prosthesis 100 and a delivery system 400. The heart valve prosthesis 100 and other embodiments disclosed herein provide more reliable valve leaflet capture than in other prior device and methods. The components and assemblies have less, minimally, or a-traumatic device materials and configurations to enhance safety. As discussed further below, the devices and methods disclosed herein provide improved treatment for severe degrees of tricuspid valve regurgitation. The heart valve prosthesis 100 includes an atrial member 212 with a cover 223 and a ventricular member 112 configured to avoid chordea in delivery and assembly and to provide atraumatic interaction leaflet tissue. These components and assemblies, discussed in greater detail below, are informed by a physiologic approach to treating tricuspid valve regurgitation which incorporates an understanding of the tricuspid anatomy.

### I. HEART VALVE REGURGITATION AND ITS ENDOVASCULAR TREATMENT

As discussed above, poor coaptation of valve leaflets is a major problem. FIGS. 1A-1C illustrate this problem and a technique for correcting it. FIG. 1A is a schematic representation of a heart 10 in normal diastole. The heart 10 consists of four chambers with the tricuspid valve 12 located between the right atrium 14 and the right ventricle 16. FIG. 1B illustrates the heart 10 in normal systole, showing that the leaflets LF1, LF2, LF3 of the tricuspid valve 12 are contacting each other where they meet. This leaflet contact seals the right atrium 14 from the right ventricle 16 along the length of the leaflets, sometimes called a line of coaptation.

FIG. 1C schematically illustrates a malfunctioning tricuspid valve 12. This view shows the tricuspid valve from above, e.g., the view from the right atrium. FIG. 1C illustrates that during systole there are large gaps G between the leaflet edges, with a complete lack of coaptation along these leaflets. FIG. 1D illustrates how heart valve prostheses described herein improve the valve function in systole. In this view, the heart valve prosthesis 100 is disposed within and captures the leaflets of the tricuspid valve. Specifically, the heart valve prosthesis 100 is applied such that each leaflet is engaged by a frame member or petal of the atrial member 212 of the heart valve prosthesis 100. Also, each leaflet of the valve is engaged by arms of the ventricular member 112. The second end 136 of the arms and the frames 222 or petals 236 of the atrial member 212 grip the first heart leaflet LF1, LF2, LF3 to reduce or eliminate regurgitation through the valve 12. FIG. 1D presents a simplified version of the heart valve prosthesis 100, showing three frames 222 and three arms 124. The heart valve prosthesis 100 can include two frames 222 and two arms 124 or more than three frames 222 and three arms 124, e.g., any number of arms including four, five six, seven, eight, nine, ten, eleven, twelve or more than twelve frames 222 and four, five six, seven, eight, nine, ten, eleven, twelve or more than twelve arms 124, or any combination of these numbers of frames and arms. In some embodiments, there are more frames 222 than arms 124, e.g., twelve frames 222 and six arms 124 or other combinations of frames and arms from the foregoing numbers of each of these components to form the heart valve prosthesis 100. In some embodiments, there are more arms 124 than frames 222, e.g., twelve arms 124 and six frames 222 or other combinations of frames and arms from the foregoing numbers of each of these components to form the heart valve prosthesis 100. The heart valve prosthesis 100 has a cover 223 blocks flow through a substantial portion of the valve 12 even if the uncovered portions of the valve along the lines of coaptation do not close. The result is that the volume of backflow (regurgitation) is reduced or prevented in systole. The leaflets can still separate in diastole sufficiently to allow blood to flow from the right atrium to the right ventricle.

The present disclosure illustrates a closure of at least a central zone of leakage or cause the uncovered portions of the valve 12 close in systole. The resulting repaired valve is much more capable of providing healthy blood flow through the heart 10.

### II. HEART VALVE REPAIR SYSTEM

Having illustrated one example of treating the tricuspid valve 12 with an embodiment of the heart valve prosthesis 100, additional details of various embodiments of the heart valve prosthesis 100 and a treatment system 50 for treating patients with heart valve regurgitation will now be discussed. FIG. 1 illustrates the treatment system 50 that includes a heart valve prosthesis 100 coupled with a delivery system 400. The heart valve prosthesis 100 is particularly well suited for treating tricuspid valve regurgitation, as described above.

The heart valve prosthesis 100 includes a ventricular member 112 and an atrial member 212. The ventricular member 112 is configured to be advanced by the delivery system 400 into a ventricle of a heart, e.g., the right ventricle. The atrial member 212 is configured to be advanced by the delivery system 400 into an atrium of a heart, e.g., the right atrium. A further discussion of the delivery system 400 is set forth below.

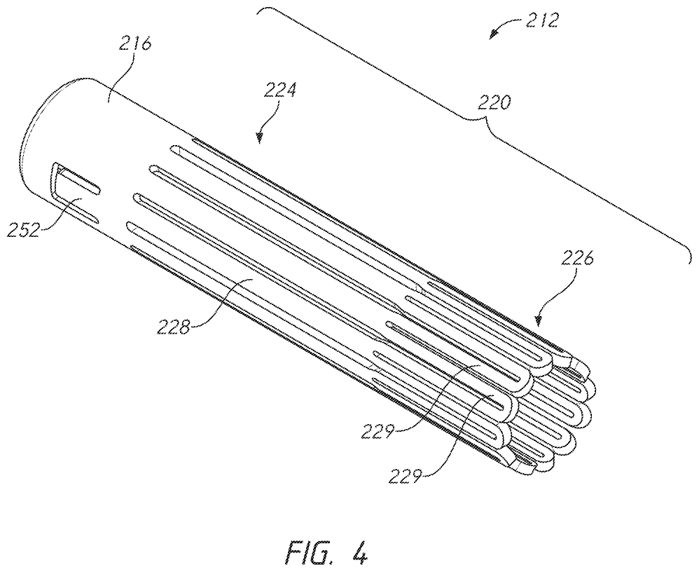

The heart valve prosthesis 100 is further illustrated in FIGS. 2-5. FIG. 2 show a collapsed configuration of the ventricular member 112. The ventricular member 112 includes a hub 116, which is sometimes referred to herein as a first hub 116. The ventricular member 112 includes one or more slots 120 formed through the first hub 116. The slots 120 are one example of a connection feature of ventricular member 112 of the heart valve prosthesis 100. The ventricular member 112 includes array of arms 124. The ventricular member 112 can include at least two, three, four, five, six, seven, eight, nine, ten, eleven or twelve arms 124. The ventricular member 112 can include no more than two, three, four, five, six, seven, eight, nine, ten, eleven or twelve arms 124. The ventricular member 112 can include only two, only three, only four, only five, only six, only seven, only eight, only nine, only ten, only eleven or only twelve arms 124. The arms 124 have a first end 132 connected to the first hub 116. The arms 124 have a second end 136 opposite the first end 132. FIG. 2 shows that in one embodiment, the array of arms 124 can include some arms 124 that are longer than other arms. A shorter arm 124 can have a first end 132′ and a second end 136′. The second end 136′ can be located closer to the first hub 116 than is the second end 136 of the longer arms 124. Each arm 124 of the array of arms 124 can include an elongate body 130 disposed between the first end 132 and the second end 136.

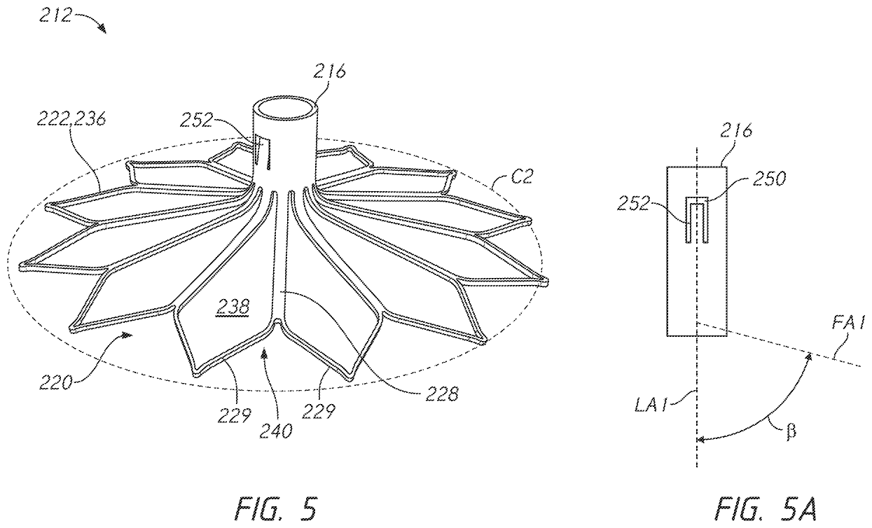

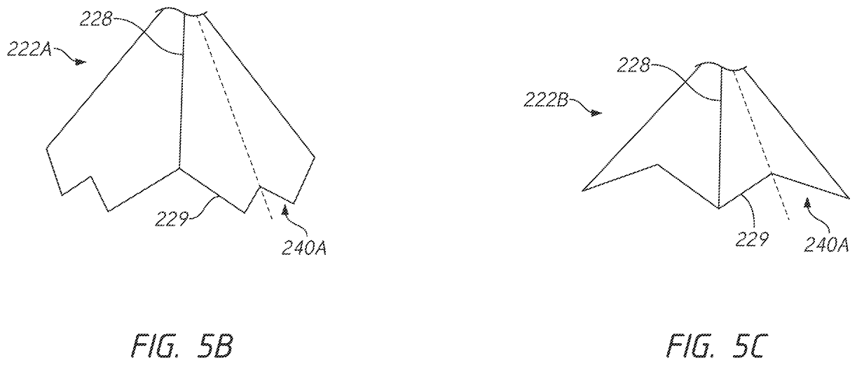

FIGS. 3 and 3A show that the first end 132 of one or more arm, e.g., each arm, of the ventricular member 112, can be deflected towards the first hub 116 in a free state 140. For example, the first hub 116 can be arranged along a lumen axis LA1. The lumen axis LA1 can be an axis in the center of a lumen within the first hub 116. FIG. 3A shows the first hub 116 and a single arm 124 for simplicity. The arm 124 has the first end 132 and the second end 136 as discussed above and extends along the elongate body 130. The first end 132 can be the end of the elongate body 130 coupled or attached to the first hub 116. The first end 132 can be coupled or attached to a distal end of the first hub 116. The elongate body 130 can include a first curved segment 130a adjacent to or extending from the first end 132 and a second segment 130b adjacent to or extending from the first segment 130a. The second segment 130b can be a straight segment. The second segment 130b can be curved. In one embodiment, the second segment 130b is curved in the same direction as the first segment. If curved, the second segment 130b can have a much larger radius of curvature than the first segment 130a. The radius of curvature can be about 0.25 mm, about 0.5 mm, about 1 mm, about 1.5 mm, about 2 mm, about 2.5 mm, or in a range including any of the foregoing dimensions as end points. The size of the radius of curvature can be a balance between being as small as possible but also large enough to minimize strains during manufacturing or implantation. An arm axis AA1 can be defined along the elongate body 130. In one embodiment, the arm axis AA1 can be a straight line aligned with or parallel to the longitudinal axis of the second segment 130b. In one embodiment, the arm axis AA1 can be a tangent line to the second segment 130b of the elongate body 130. An angle of deflection a can be defined between the lumen axis LA1 and the arm axis AA1. The angle of deflection a can be about 5 degrees, about 10 degrees, about 15 degrees, about 20 degrees, about 25 degrees, about 30 degrees, about 35 degrees, about 40 degrees, about 45 degrees, or within a range defined between any of the foregoing angles.

The angle of deflection a is such that a projection of the second end 136 of the elongate body 130 toward the first hub 116 is closer to a proximal end of the first hub 116 than is a projection of another part of the elongate body 130. A projection of the second end 136 of the elongate body 130 toward the first hub 116 can be disposed closer to a proximal end of the first hub 116 than is an end of the second segment 130b adjacent to the first segment 130a. A projection of the second end 136 of the elongate body 130 toward the first hub 116 can be disposed closer to a proximal end of the first hub 116 than is the first segment 130a. A projection of the second end 136 of the elongate body 130 toward the first hub 116 can be disposed closer to a proximal end of the first hub 116 than is the first end 132.

FIGS. 3 and 3A illustrate that the slots 120 can be an elongate, e.g., rectangular, opening in the side surface of the first hub 116. The slots 120 can be elongate along the direction of the lumen axis LA1. The slots 120 can have axial edges aligned with the lumen axis LA1 and circumferential edges that follow the curvature of the first hub 116. As discussed further below, the slots 120 can engage connection features of the atrial member 212 when the heart valve prosthesis 100 is assembled within a heart of a patient.

The ventricular member 112 can be formed in any suitable manner. In one embodiment, the ventricular member 112 is formed from a tubular body. The elongate body 130 of each of the arms 124 can be formed by cutting or removing material between an elongate body 130 and one or more adjacent elongate bodies 130. FIG. 2 shows that the ventricular member 112 can have a tubular configuration when in an unexpanded or compressed state. The outward facing surfaces of the elongate body 130 can be aligned with a projection of the outer surface of the first hub 116. The outward facing surfaces of the elongate body 130 can be disposed radially inward of a projection of the outer surface of the first hub 116. These configurations facilitate the use of a cylindrical catheter body of the delivery system 400 to maintain the arms 124 in an unexpanded or compressed state for delivery and thereafter extend the ventricular member 112 out of the cylindrical catheter body for expansion in a ventricle, as discussed further below.

In one embodiment, the ventricular member 112 can be formed using or can include a shape memory material. Such a material can enable the arms 124 to self-expand from the configuration of FIG. 2 into the configuration of FIG. 3. One such material is a nickel-titanium alloy know as Nitinol. A shape memory material that is configurated to be highly elastic (e.g., super-elastic) can allow the configuration of FIG. 3 to be attained by removing a constraint (e.g., an outer catheter body of the delivery system 400) from a position radially outward of the arms 124. Once the arms 124 are unconstrained, the arms can move from the compressed state of FIG. 2 to the expanded state of FIG. 3. Shape memory materials also can be configured to be actuated by ambient temperature. The material used to form the ventricular member 112, e.g., the arms 124, can be configured such that the compressed state of FIG. 2 is provided at room temperature and the expanded state of FIG. 3 is provided by raising the temperature of the arms 124 to a higher temperature, e.g., the body temperature of the patient.

FIG. 2 shows that the arms 124 can have an atraumatic configuration. In one embodiment, an atraumatic configuration is provided by configuring the second end 136 of one or at least some (e.g., all) of the arms 124 with an atraumatic tip. The atraumatic tip can have an enlarged portion 136a and a narrowed portion 136b. The enlarged portion 136a can be the same size or width (e.g., dimension transverse to a longitudinal axis of the arms 124) and can be enlarged relative to the narrowed portion 136b. The enlarged portions 136a can be larger than the dimension transverse to a longitudinal axis of the arms 124 of the narrowed portion 136b. The enlarged portion 136a can spread the contact load to valve leaflets on the ventricular side thereof to reduce the trauma thereto. The narrowed portion 136b and the varying length of adjacent arms 124 allow for enhanced packing of the arms. In particular, the narrowed portion 136b can be configured to receive the enlarged width of the enlarged portion 136a as seen in FIG. 2. As a result, a larger number of arms 124 can be included in the ventricular member 112 of a given diameter than if the enlarged portion 136a of some of the arms 124 were not nested within the narrowed portion 136b of the adjacent arms 124.

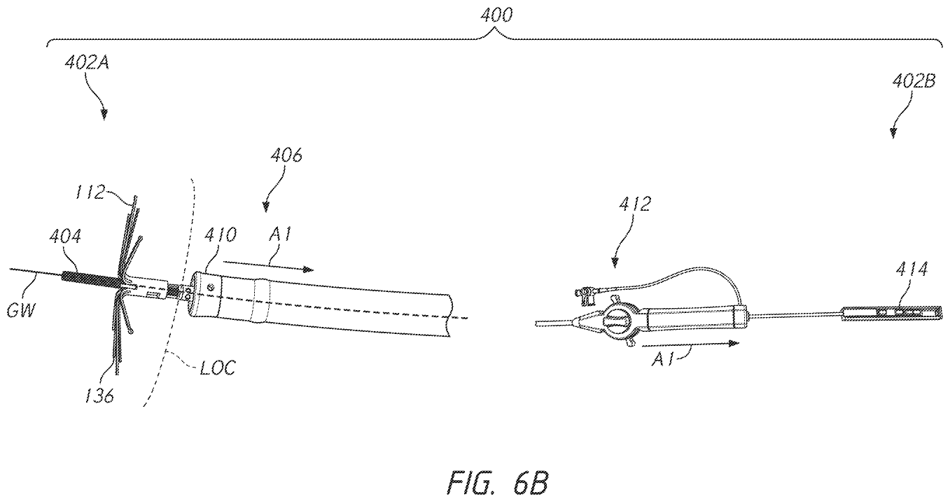

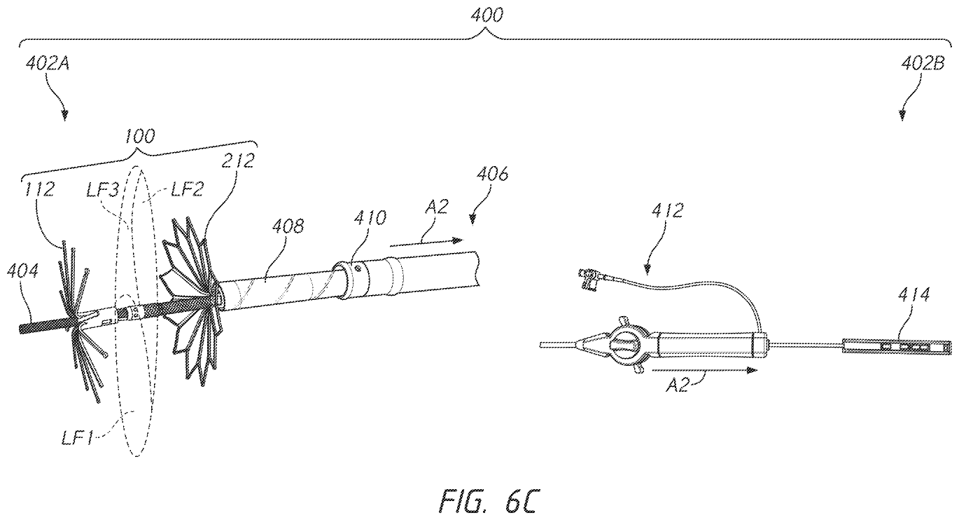

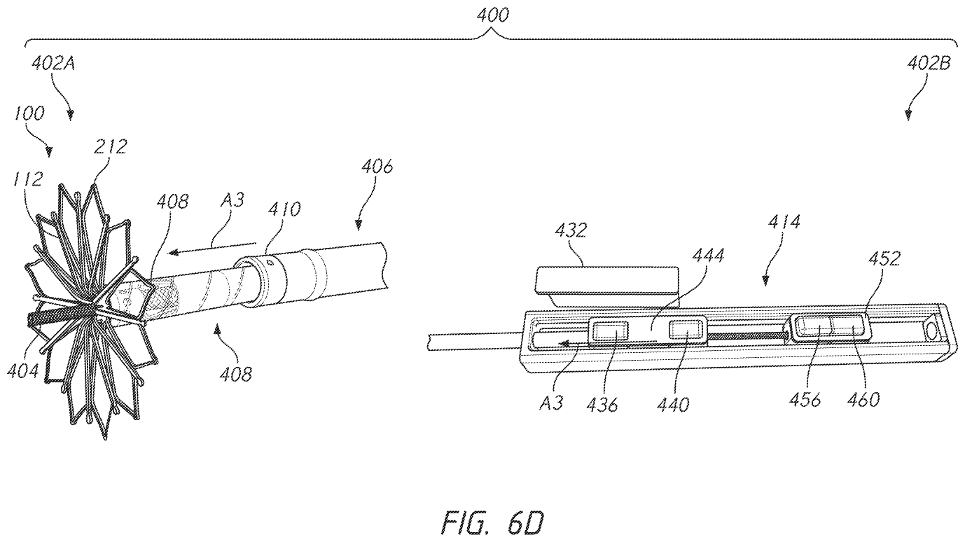

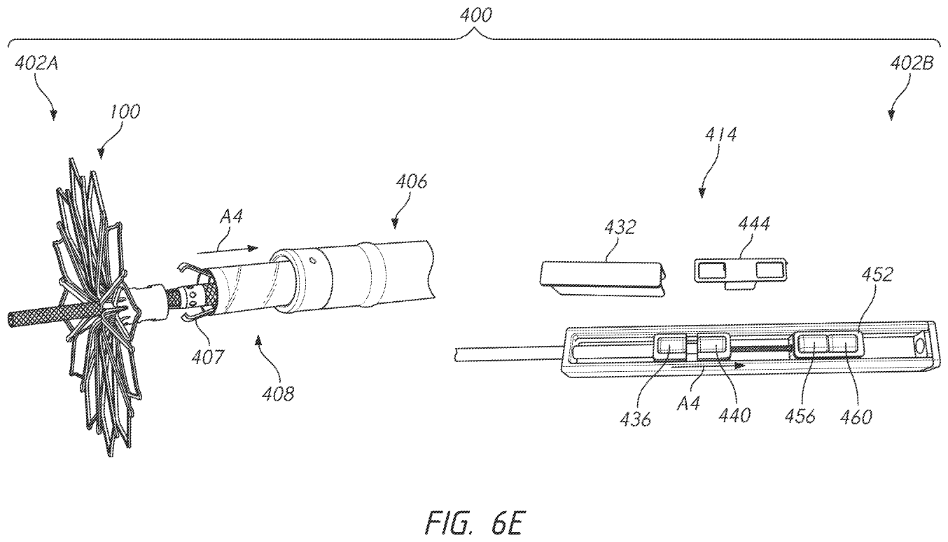

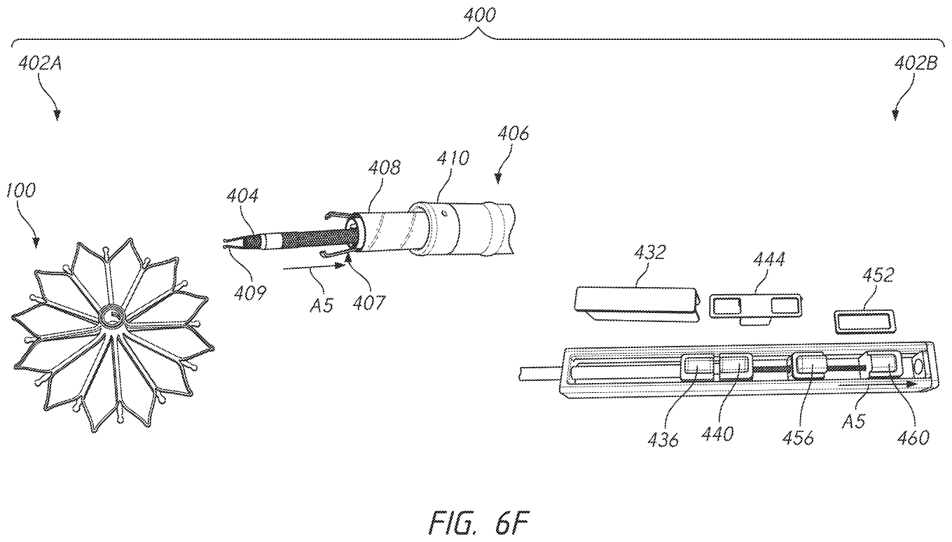

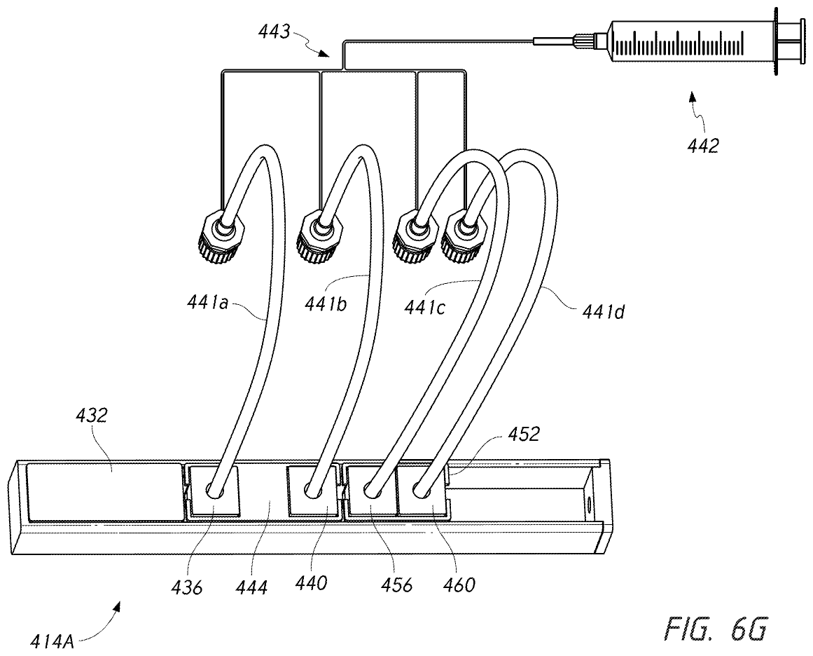

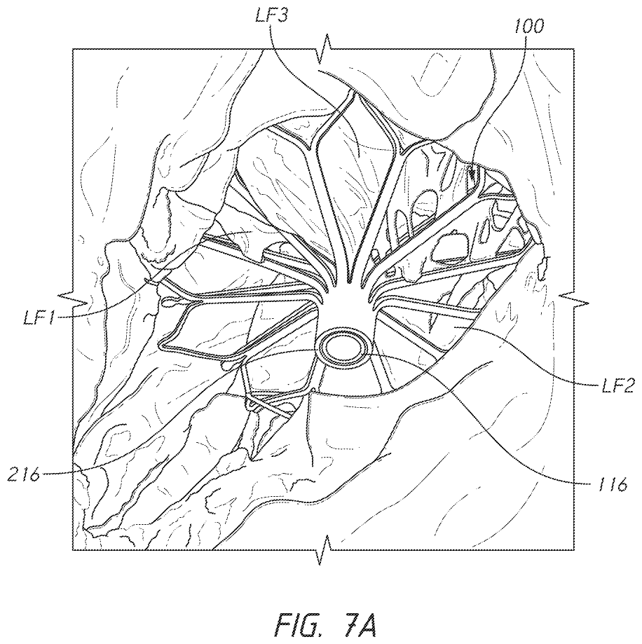

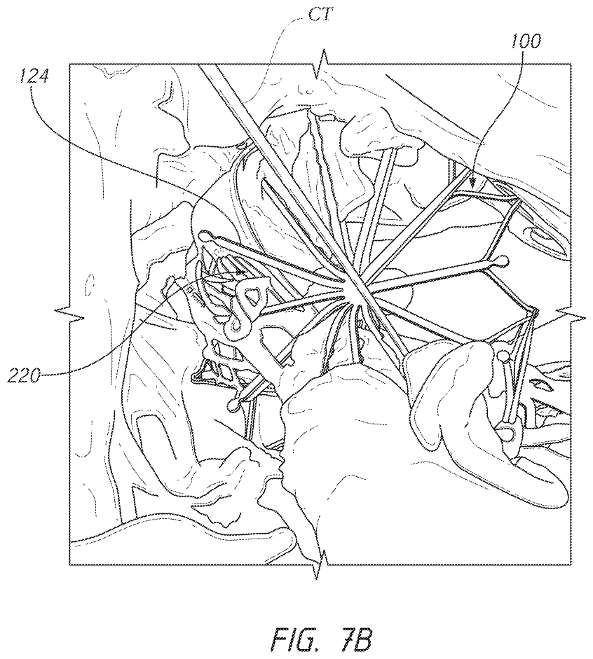

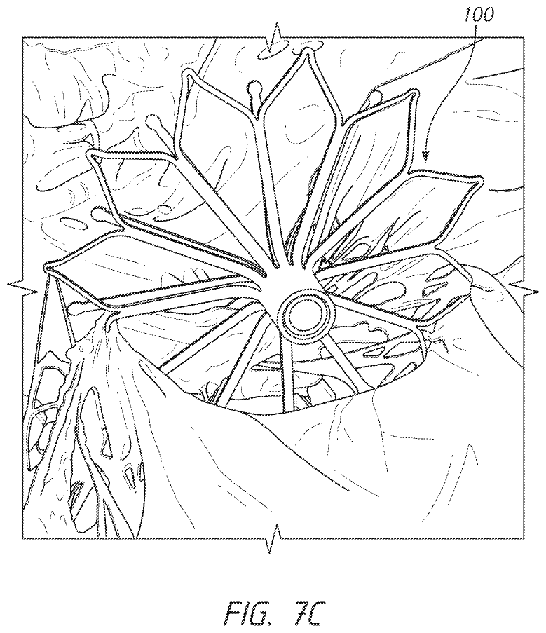

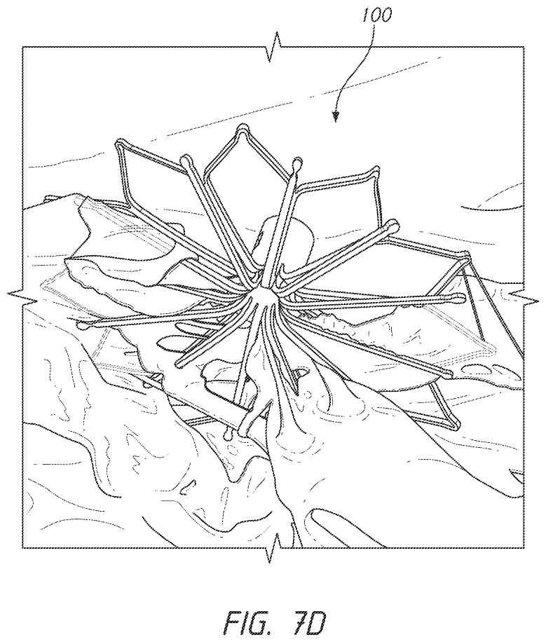

The ventricular member 112 can be configured such that the second end 136 of the arms 124 can lie on or adjacent to an arcuate profile, such as a circle C1. The circle C1 can have a diameter of about 10 mm, about 15 mm, about 20 mm, about 25 mm, about 30 mm, about 35 mm, or in a range including any of the foregoing dimensions as end points, e.g., about 15 mm to about 25 mm. The arcuate profile, e.g., circle C1 connecting the second ends 136 of the arms 124 is less than an arcuate profile, e.g., circle C2 (see FIG. 5) connecting the outer ends of petals 236 or the tips of the atrial member 212. The relative size for the circle C1 and circle C2 can be seen in FIGS. 6D and 6F and is discussed further below.

In the illustrated embodiment of FIG. 3, the second ends 136 of adjacent arms 124 are not connected to each other. One or more of the second ends 136 configured to be placed into direct contact with the first heart leaflet LF1. One or more of the second ends 136 configured to be placed into direct contact with the second heart leaflet LF2 Optionally, one or more of the second ends 136 configured to be placed into direct contact with the third heart leaflet LF3. In some embodiments, the entire elongate body 130 of adjacent arms 124 remain unconnected to the adjacent arms. The heart leaflets LF1, LF2, LF3 are connected to heart tissue by chordae tendineae, or chordae. The chordae are elongate fibrous chords spanning the ventricle. The un-connected nature of the arms 124 allows the arms to expand between adjacent chordate without becoming tangled therein. More specifically, the arms 124 can transition from extending in a direction aligned with the lumen axis LA1 to a direction corresponding to the pre-loaded configuration. This can include swinging through an angle of more than 90 degrees. The slender and un-connected configuration of the arms 124 allows the arms to move between adjacent chordae without entanglement.

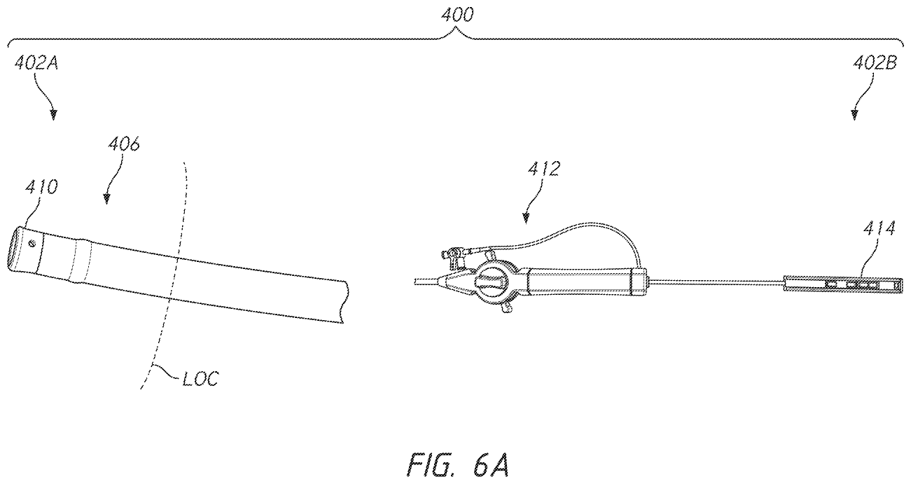

FIGS. 4 and 5 show additional details of the atrial member 212. The atrial member 212 is configured to be advanced into an atrium and to extend across a line of coaptation LOC of a first heart leaflet LF1, a second heart leaflet LF2, and/or a third heart leaflet LF3. The atrial member 212 is separate from and moveable relative to the ventricular member 112 prior to connecting or engaging the first hub 116 to a second hub 216 of the atrial member 212 to assemble the heart valve prosthesis 100. The engagement of the first hub 116 and the second hub 216 causes the arms 124 of the ventricular member 112 to be pressed into concavities of the atrial member 212. The concavities can be disposed at an outer periphery of the atrial member 212.

The atrial member 212 includes a frame array 220 including a plurality of frames 222 that can be at least partially, e.g., entirely enclosed by a cover 223. The cover 223 can include a membrane formed of a material such as ePTFE or Dacron. By positioning the cover 223 on the atrial side of the valve (on the atrial member 212), the cover 223 will not interfere with the chordae. The cover 223 and the frame array 220 provide a closed cell frame structure that creates more consistent loading around the perimeter of the atrial member 212. The cover 223 can be used to seal the portion of the valve over which the cover and the frames 222 extend. In some cases the cover 223 promotes endothelialization over the atrial member 212. The cover 223 can cause some load to be applied between the tips of the frames 222. The cover 223 can partially cover concavities between the frames 222 (discussed below) and can receive tips of the arms 124 of the ventricular member 112 with valve tissue sandwiched therebetween in some locations upon deployment.

The frames 222 can be configured as petals 236. The petals 236 can include a repeating pattern of structures about the circumference. The petals 236 can be positioned edge to edge. The petals 236 can share a common edge or strut. The petals 236 can be symmetrical about a bi-secting radius of each petal. In some embodiments, the petals 236 are symmetrical about a radius extending from a center of an inner portion of the petals 236 adjacent to the second hub 216 to a tip of the petals 236. The tip of the petals 236 can be a radially outermost portion of each petal. Where the cover 223 is provided, it can extend across the structure of the petal enclosing some or each petal. The cover 223 can have an arcuate, e.g., circular, outer periphery as shown in FIG. 1. The cover 223 can span between tips of the petals 236 at the periphery of the atrial member 212.

FIGS. 4 and 5 show that each of the frames 222 can have a base portion 224 and an outer portion 226. The base portion 224 of each frame 222 can include one or more shared struts 228. The shared struts 228 can have opposing circumferential edges bounding a portion of adjacent frames 222. A single frame 222 can be bounded by two adjacent shared struts 228. A single frame 222 can be bounded by dedicated strut portions 229. A single frame 222 can include a first shared strut 228 and a first dedicated strut portion 229 extending from an outer end of the first shared strut 228 to a tip of the frame 222. The single frame 222 can further be bounded by a second dedicated strut portion 229. The second dedicated strut portion 229 can extend from the tip of the frame 222 to an outer end of a second shared strut 228. The second shared strut 228 is disposed adjacent to the first shared strut 228 opposite a radius extending to the tip of the frame 222. The first and second shared struts 228 can be coupled at inner ends to the second hub 216 or to another frame member that is coupled to the second hub 216.

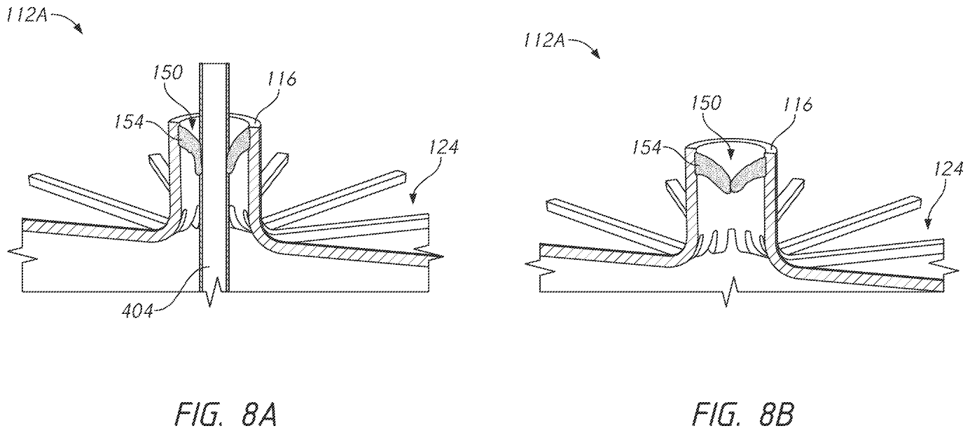

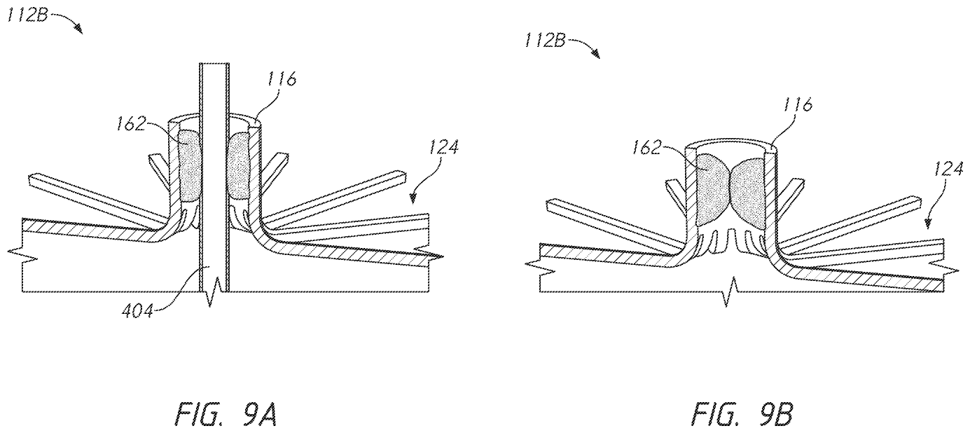

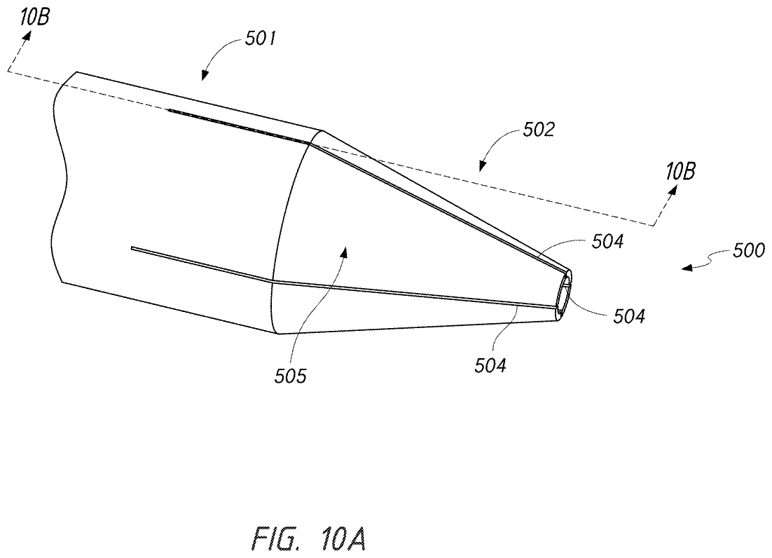

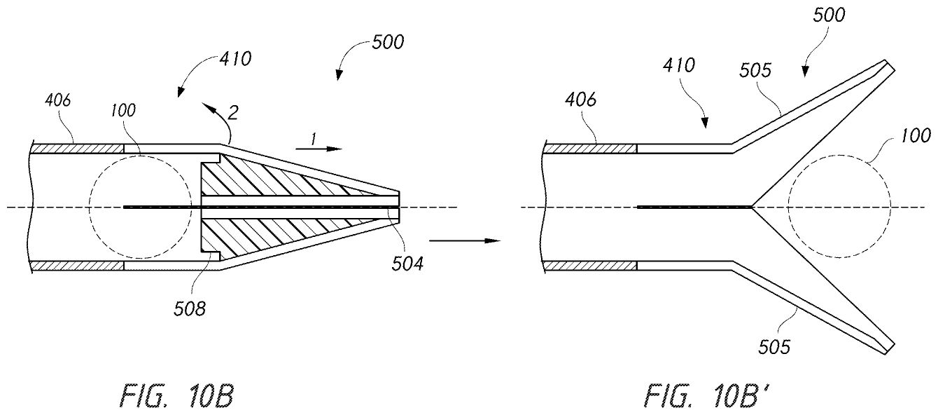

Each frame 222 encloses an approximately quadrilateral shaped area 238 in one embodiment. As discussed above, adjacent frames are connected by shared struts 228. Adjacent frames 222 form a concavity 240 therebetween. The concavity 240 provides an area for receiving or nesting of the arms 124 in the atrial member 212. The atrial member 212 is separate from and moveable relative to the ventricular member 112 prior to the first hub 116 and the second hub 216 being engaged. The concavity 240 can be centered on a longitudinal axis of the shared struts 228 in one embodiment. In an engaged configuration the arms 124 of the ventricular member 112 are pressed into the concavity 240 of the atrial member 212. This relationship is shown in FIGS. 6D and 6F and 7B-7D.

FIG. 5 shows that the quadrilateral shaped area 238 can have a kite shape. For example, in some embodiments the shared struts 228 can have a length that is greater than the length of the dedicated strut portions 229. In some embodiments the shared struts 228 extend away from a central radial axis of the frames 222 by a first angle and the dedicated strut portions 229 extend away from the central radial axis by a second angle that is greater in magnitude than the first angle. The kite shaped area can be defined by a first isosceles triangular shaped area and a second isosceles triangular shaped area. The first isosceles triangular shaped area can be defined by an axis extending between outer ends of the shared struts 228 and by the two shared struts 228 of an individual frame 222. The second isosceles triangular shaped area can be defined by the axis extending between outer ends of the shared struts 228 and by the dedicated strut portions 229 of an individual frame 222. The first isosceles triangle can be disposed between the second isosceles triangle and the second hub 216. The first isosceles triangular shaped area can have a first height greater than a second height of the second isosceles triangle. The first height can be in a range of about 5 mm to about 9 mm. The second height can be in a range of about 2 mm to about 6 mm. The height of the first isosceles triangular shaped area can be about 20 percent, about 30 percent, about 40 percent, about 50 percent, about 60 percent, about 70 percent, about 80 percent, about 90 percent, about 100 percent, about 150 percent, about 200 percent, or in a range including any two of the foregoing percentages listed as end points larger than the height of the second first isosceles triangular shaped area. The height of the first isosceles triangular shaped area can the same or less than the height of the second first isosceles triangular shaped area. The magnitude of the height of the first isosceles triangular shaped area can be about twice the magnitude of the length of the base of the first isosceles triangular shaped area. The magnitude of the height of the second isosceles triangular shaped area can be about the same as the magnitude of the length of the base of the second isosceles triangular shaped area.

FIG. 5A is a schematic diagram illustrating a preloaded state of the atrial member 212. In the preloaded state, the frames 222 are oriented toward the location of the leaflets when the atrial member 212 is positioned or implanted in the heart. The atrial member 212 can provide that the frames 222 extend from a distal end of the second hub 216 outward and distally. In one embodiment, the amount of the extent in the distal direction can be defined by an angle of deflection B. The angle of deflection B can be about 85 degrees, about 80 degrees, about 75 degrees, about 70 degrees, about 65 degrees, about 60 degrees, about 55 degrees, about 50 degrees, about 45 degrees, or within a range defined between any of the foregoing angles. The magnitude of the angle of deflection B can be the same as the magnitude of the angle of deflection a of the arms 124. The magnitude of the angle of deflection B can be greater than the magnitude of the angle of deflection a of the arms 124. The magnitude of the angle of deflection B can be less than the magnitude of the angle of deflection a of the arms 124.

FIG. 3A shows that the arms 124 are bent or are inclined toward the first hub 116 when the ventricular member 112 is disengaged from the atrial member 212. FIG. 3A illustrates a preloaded state in which the arms 124 are oriented toward the location of the leaflets when the ventricular member 112 is positioned or implanted in the heart. FIG. 5A shows that the frames 222 or the petals 236 are bent or are inclined away from the second hub 216 (e.g., toward the leaflet location) when the atrial member 212 is disengaged from the ventricular member 112. The arms 124 and the base portion 224 are each elastic, e.g., formed of a nickel-titanium alloy (e.g., Nitinol) or similar highly elastic material, such that the arms 124 and the base portion 224 apply a load when they are deflected away from the at rest states of FIGS. 3A and 5A. If the arms 124 are deflected to higher angles α the arms 124 will apply a proximally oriented load to the leaflets of the valve when implanted. If the frames 222 are deflected to higher angles ß the frames 222 will apply a distally oriented load. As discussed further below, in one embodiment the process of assembling the first hub 116 of the ventricular member 112 to the second hub 216 of the atrial member 212 will cause the arms 124 to deflect to higher angles α and the frames 222 to deflect to high angles ß. Thus, in this embodiment, both the arms 124 and the frames 222 will apply compression loads on one or more of the first heart leaflet LF1, the second heart leaflet LF2, and optionally the third heart leaflet LF3 compressed therebetween. In other embodiments, one of the arms 124 and the frames 222 are more deflectable or one of the arms 124 and the frames 222 is not deflectable under the loads involved in assembling the heart valve prosthesis 100. Thus, the load applied to one side of the leaflets LF1, LF2, LF3 can be different from the load applied to the other side of the leaflets LF1, LF2, LF3.

FIGS. 5B and 5C show additional configurations of frames 222A, 222B. The frames 222A have a concavity 240A that is centered on a central radial axis (dashed line) of the frames. As with the frames 222, the frames 222A are formed by radially outward extending dedicated strut portions 229. The concavity 240A can be formed by undulating shape of portions of the dedicated strut portions 229 disposed away from the shared struts 228. The concavity 240A can be located between the adjacent dedicated strut portions 229 of the frames 222A. The frames 222B have a concavity 240B that is centered on a central radial axis (dashed line) of the frames 222B. As with the frames 222, the frames 222B are formed with dedicated strut portions 229 that extend from outer ends of the shared struts 228. The dedicated strut portions 229 of the frames 222B extend radially inward from the outer ends of the shared struts 228 to form the concavity 240B along the central radial axis of the frames 222B. The concavity 240B can be formed by straight inwardly extending dedicated strut portions 229 of the frames 222B.

FIG. 5 shows that the frames 222 of the atrial member 212 can have tips disposed on an arcuate periphery, e.g., a circle C2. The circle C2 can be configured to be larger than the circle C1 extending between the ends of the arms 124 of the ventricular member 112. The location of the concavity 240, concavity 240A, or the concavity 240B can be disposed at a radial position that is less than the radius of circle C1. Thus the tips of the arms 124 can be disposed in an annular band between the circumference of the circle C1 and the circumference of the circle C2. The circle C2 can have a diameter of about 15 mm, about 20 mm, about 25 mm, about 30 mm, about 35 mm, about 40 mm, or in a range including any of the foregoing dimensions as end points.

The atrial member 212 also can have connection feature. In one embodiment, a tang (or a plurality of tangs) 252 is (or are) configured for locking into the slot(s) 120 of the first hub 116 to engage the second hub 216 with the first hub 116 when the atrial member 212 and the ventricular member 112 are assembled. The tangs 252 can be configured to engage a connection feature of the ventricular member 112, e.g., the slots 120. In one embodiment, the first hub 116 is received in the second hub 216. The tangs 252 are biased inwardly and will deflect inward into the slots 120 when the tangs 252 are aligned with the slots 120. In another embodiment, the second hub 216 is received in the first hub 116. The tangs 252 are biased outwardly and will deflect outward into the slots 120 when the tangs 252 are aligned with the slots 120.

The heart valve prosthesis 100 preferably has a centering feature to cause the connection features of the ventricular member 112 and the atrial member 212 to be aligned with each other upon engagement of the members with each other. In one example, as the arms 124 are moved into the concavity 240 the sloping side surfaces of the dedicated strut portions 229 guide the arms 124 into the radially inner-most portion of the concavity 240. This position corresponds to an alignment of the tangs 252 with the slots 120. For example, in one embodiment, the delivery system 400 is configured such that the atrial member 212 is held rotationally stationary and the frames 222 or petals 236 can apply a load to the arms 124 which thereby apply a moment to the first hub 116 to rotate the first hub 116 relative to (e.g., within or over) the second hub 216 to rotate the slots 120 into rotational alignment with the tangs 252. In another embodiment of the delivery system 400 the ventricular member 112 is maintained rotationally stationary and the arms 124 apply a force to the frames 222 or to the petals 236 which thereby apply a moment to the second hub 216 to rotate the second hub 216 relative to (e.g., within or over) the first hub 116 such that the tangs 252 move into rotational alignment with the slots 120. Thus, the arms 124 can act as a centering feature, the concavity 240 can also act as a centering feature or the arms 124 and the concavity 240 can also act as a centering feature of the heart valve prosthesis 100.

As with the ventricular member 112, the atrial member 212 can be compressed into a cylindrical configuration as seen in FIG. 4. The cylindrical configuration allows the atrial member 212 to be disposed in a catheter body of the delivery system 400 as discussed below. As discussed above, the atrial member 212 can be formed of an elastic material, e.g., a nickel-titanium material such as Nitinol, to be able to self-expand to the configuration of FIG. 5.

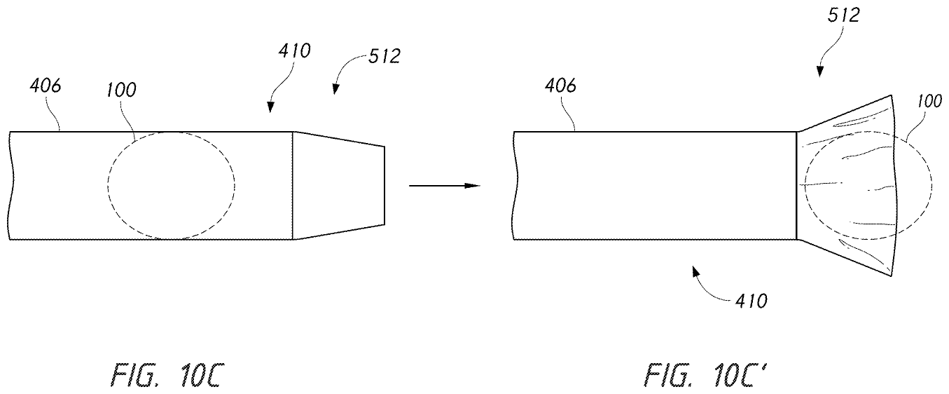

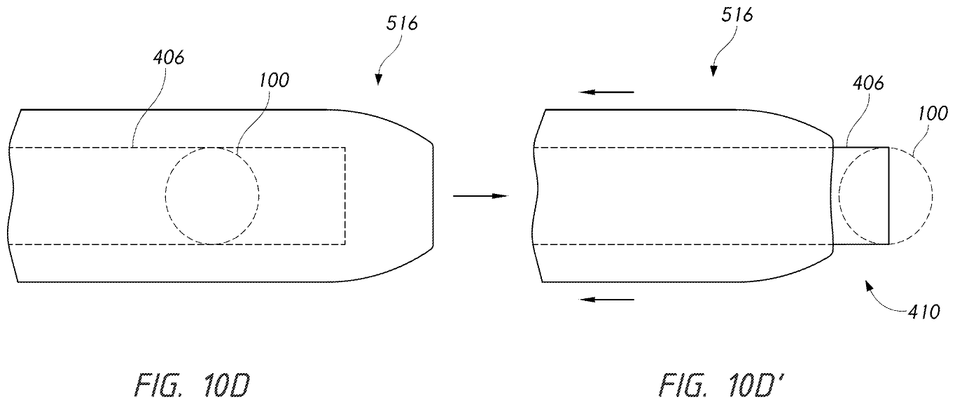

FIGS. 8A-9B illustrates additional embodiments of the heart valve prosthesis that can incorporate a plug or other flow control for a heart valve prosthesis. The prosthesis can include a ventricular member 112A or a ventricular member 112B illustrated thereby. The ventricular member 112A and the ventricular member 112B can be similar to the ventricular member 112 except as described differently below.

The ventricular member 112A can include a deflectable flow control member 150 disposed within the first hub 116. The deflectable flow control member 150 can be configured as a one way valve with deflectable flaps 154 configured to span the lumen of the first hub 116. The deflectable flaps 154 can be deflected as shown in FIG. 8A to accommodate an inner shaft 404, which can be part of the delivery system 400 as discussed further below. The deflection of the deflectable flaps 154 allows the inner shaft 404 to be disposed through the ventricular member 112A during delivery. The inner shaft 404 can be removed during or at the end of deliver and assembly of the heart valve prosthesis 100 incorporating the ventricular member 112A. FIG. 8B shows that after the inner shaft 404 is removed from the lumen of the first hub 116 the deflectable flaps 154 extend across the lumen and seal against each other. The pressure on the distal side of the ventricular member 112A (i.e., in the right ventricle) will exceed that of the pressure on the proximal side of the ventricular member 112A and when that condition is present the deflectable flaps 154 will remain closed. The deflectable flow control member 150 is advantageous in that the valve would allow access through the heart valve prosthesis 100 for reintroducing the inner shaft 404 or performing other procedures through the heart valve prosthesis 100.

FIGS. 9A-9B illustrate the ventricular member 112B in more detail. A compressible flow control member 162 is provided within the lumen of the first hub 116. The compressible flow control member 162 can be a compressible foam material, a woven Dacron material, or other similar material. The compressible flow control member 162 can be provided with a small opening allowing the inner shaft 404 to be passed therethrough. After delivery of the ventricular member 112B the inner shaft 404 can be removed from the lumen of the first hub 116 of the ventricular member 112B. The pressure in the ventricle will cause the small opening to collapse and to close, plugging the lumen of the first hub 116. The compressible flow control member 162 is advantageous in that after the inner shaft 404 is removed a complete closure of the lumen through the first hub 116 (and through the heart valve prosthesis 100) is provided regardless of the pressures on opposite sides of the ventricular member 112B. The compressible flow control member 162 can initially have an opening, e.g., not be fully closed when the inner shaft 404 is not disposed therethrough. A small size opening may become occluded with tissue over time. In some cases the ventricular member 112 can be provided without any collapsible flow control member and yet can be closed due to tissue growth over time.

### III. DELIVERY SYSTEMS AND METHODS

FIGS. 1 and 6A-6F illustrate various embodiments of the delivery system 400 for delivering and assembling the heart valve prosthesis 100 within a patient's heart, e.g., in the tricuspid valve between the right atrium and ventricle. The delivery system 400 is configured to be delivered over a guidewire GW as seen in FIGS. 1 and 6A-6E. In some techniques, the guidewire GW can be advanced into the ventricle and held at rest (e.g., parked) in the ventricle to provide a rail for rapid and accurate advancement and deployment of the prosthesis 100. In other techniques, a guide catheter (not shown) can be advanced into the ventricle and held at rest in the ventricle to provide a rail for rapid and accurate advancement and deployment of the prosthesis 100. A guide catheter can be used alone or in combination with a guidewire to facilitate deployment. The delivery system 400 includes a distal portion 402A and a proximal portion 402B. An outer sheath 406 can extend to a distal tip 410. The proximal portion 402B can include a guide handle 412 and a delivery handle 414. The guide handle 412 can be coupled with a proximal end of the outer sheath 406. The delivery handle 414 can extend through the guide handle 412 and through the outer sheath 406 to components of the heart valve prosthesis 100.

FIG. 6A shows an aspect of a method of using the delivery system 400 to deliver the heart valve prosthesis 100. The guide handle 412 can be manipulated to advance the outer sheath 406 through the venous vasculature over a guidewire to the heart. The outer sheath 406 can be advanced into the right atrium. Continued advancement of the outer sheath 406 can move the distal tip 410 across the line of coaptation LOC of the tricuspid valve of the patient into the right ventricle. This is the position illustrated in FIG. 6A. The delivery handle 414 controls movement and relative position of the components of the heart valve prosthesis 100, e.g., during movement of the guide handle 412 and the outer sheath 406. The delivery handle 414 can be held stationary while the guide handle 412 is pulled back to expose the implant. In various methods, the heart valve prosthesis 100 is placed in one of the peripheral regions of the tricuspid valve.

FIG. 1D shows that the heart valve prosthesis 100 can be implanted between the first heart leaflet LF1 and the second heart leaflet LF2. The placement of the heart valve prosthesis 100 in FIG. 1D can be such that the heart valve prosthesis 100 is not disposed across the gap G between the first heart leaflet LF1 and the third heart leaflet LF3 unaffected. The placement of the heart valve prosthesis 100 in FIG. 1D can be such that the heart valve prosthesis 100 is not disposed across the gap G between second heart leaflet LF2 and the third heart leaflet LF3 unaffected. In other techniques, the heart valve prosthesis 100 can be placed between the first heart leaflet LF1 and the third heart leaflet LF3. In other techniques, the heart valve prosthesis 100 can be placed between the second heart leaflet LF2 and the third heart leaflet LF3. The appropriate position of the heart valve prosthesis 100 can be determined prior to or during the procedure, e.g., using an echocardiogram. The appropriate position of the heart valve prosthesis 100 can be achieved by manipulating the distal tip 410 and a distal length of the outer sheath 406 within a peripheral region of a portion of the tricuspid valve between two of the three leaflets.

FIG. 6B shows that relative movement between the outer sheath 406 and the ventricular member 112 can expose the ventricular member in the right ventricle. The outer sheath 406 can be moved proximally by withdrawing the guide handle 412 as indicated by the arrow A1. The delivery handle 414 can be held stationary while the guide handle 412 is moved as indicated by the arrow A1 in one technique. As the outer sheath 406 moves proximally, the distal tip 410 is withdrawn proximally until the distal tip 410 is disposed proximal of the ventricular member 112. The distal tip 410 can be withdrawn into the right atrium while the delivery system 400 maintains the ventricular member 112 in position, e.g., the ventricular member can be maintained at a same position while the distal tip 410 is moved proximally. In another technique the guide handle 412 can be held stationary and the delivery handle 414 can be moved distally to advance the ventricular member 112 distally out of the distal tip 410 of the outer sheath 406.

As discussed above, the ventricular member 112 can include arms 124 that can be configured to self-expand into the position or configuration seen in FIG. 6B. The arms 124 can be arranged such that the second ends 136 thereof are not connected to each other. The arms 124 are thus slender members that can expand between the network of chordae that transverse the right ventricle to move the leaflets LF1, LF2, LF3 as the heart beats. This configuration of the ventricular member 112 mitigates or eliminates the risk of entanglement between the ventricular member 112 and the chordae. In one form of the delivery system 400 the rotational position of the delivery handle 414 can be maintained as the outer sheath 406 is moved relative to the ventricular member 112 such that the ventricular member does not rotate as it is expanded.

FIG. 6C shows a further aspect of a method of using the delivery system 400. The outer sheath 406 can be moved relative to an inner sheath 408, e.g., moved proximally as indicated by the arrow A2, until distal tip 410 is proximal of the atrial member 212. The atrial member 212 can be disposed distally of the inner sheath 408 such that when exposed, the atrial member 212 can expand within the atrium. Proximal movement along the arrow A2 can be caused by corresponding movement of the guide handle 412 as indicated by the arrow A2. FIG. 6C shows that the ventricular member 112 and the atrial member 212 can be expanded within the heart prior to being engaged with each other. The ventricular member 112 can be preloaded, e.g., with the arms 124 deflected or oriented toward the first hub 116 of the ventricular member as discussed in connection with FIG. 3A. The frame array 220 can be preloaded, e.g., with the frames 222 or petals 236 deflected or oriented away from the second hub 216 as discussed in connection with FIG. 5A. The angle of deflection or orientation of the arms 124 and the frame array 220 can be greater in the state of FIG. 6C than when the ventricular member 112 and the atrial member 212 are engaged with each other.

FIG. 6D shows a technique for grasping one or more, e.g., two or three, leaflets. The delivery handle 414 can be modified by removing a first lockout 432 from a first slider 436 and a second slider 440. The first lockout 432 is configured to block movement of the first slider 436 and the second slider 440 prior to being removed from the delivery handle 414. FIG. 6C shows that the first lockout 432 can fill a space in the delivery handle 414 in which the first slider 436 and the second slider 440 can move if the first lockout 432 is not positioned in that space. Upon removal of the first lockout 432 the first slider 436 and the second slider 440 can be moved together as indicated by the arrow A3. This movement can cause a corresponding movement according to arrow A3 moving the atrial member 212 toward and into engagement with the ventricular member 112. Following the movement of the first slider 436 and the second slider 440 the ventricular member 112 and the atrial member 212 can be engaged, trapping leaflet material therebetween. At least one of the tips (e.g., the second end 136) of the arms 124 can be received in the concavities 240 of the frame array 220. The ventricular member 112 and the atrial member 212 can still be connected to the delivery system 400 in this part of the method of using the delivery system 400. FIG. 6D shows that a second lockout 444 can maintain the relative position of the first slider 436 and the second slider 440 during the movement according to the arrow A3. FIG. 6D also shows that a third lockout 452 can be provided to maintain the relative position of a third slider 456 and a fourth slider 460, the operation of which will be discussed further below.

As discussed above the tangs 252 can be configured to engage windows, e.g., the slots 120, of the ventricular member 112. The tangs 252 can be deflected inwardly in a free state. Prior to engaging the first hub 116 with the second hub 216 (e.g., advancing the first hub 116 into the second hub 216) the tangs 252 can be deflected inwardly such that a minimum distance between the tangs 252 is less than an outer diameter of the first hub 116. The tangs 252 can be rotationally aligned with the slots 120 such that as the movement according to the arrow A3 occurs the tangs 252 can be advanced to a proximal edge of the first hub 116. As the proximal edge engages the inner side of the tangs 252 the tangs can be deflected outwardly by the first hub 116. Continued relative movement of the first hub 116 and the second hub 216 to each other can be provided, e.g., according to the arrow A3, until the tangs 252 are disposed over the slots 120. When disposed over the slots 120, the tangs 252 will no longer be held in a radially outwardly deflected position and can deflect inwardly into the slots 120 to securely connect the ventricular member 112 to the atrial member 212.

As discussed above, the concavities 240 can receive the arms 124 (in at least some cases with valve tissue therebetween) and as continued movement of the ventricular member 112 and the atrial member 212 together occurs, the concavity 240 can provide at least some rotational movement of the members to each other. This rotational movement can reduce or eliminate any misalignment of the tangs 252 to the slots 120 that could complicate engagement of these connection features.

FIG. 6E shows a further aspect of a method of using the delivery system 400. The second lockout 444 is removed allowing for relative movement of the first slider 436 relative to the second slider 440. The first slider 436 can be moved toward the second slider 440 as indicated by the arrow A4. Such movement corresponds to withdrawing a distal portion of the inner sheath 408 to allow an outer gripper 407 of the delivery system 400 to expand. Prior to expansion thereof, the outer gripper 407 is engaged with the second hub 216, e.g., with windows 250 of the second hub 216 disposed around the tangs 252. Retracting the inner sheath 408 from the outer gripper 407 can allow the outer gripper 407 to self-expand out of engagement with the window 250.

FIG. 6F shows disengagement of the heart valve prosthesis 100 from the delivery system 400. The disengagement can be achieved by removing the third lockout 452 from the delivery handle 414. The third lockout 452 can prevent relative movement of the third slider 456 to the fourth slider 460. After the third lockout 452 is removed the fourth slider 460 can be moved proximally as indicated by the arrow A5 retracting the inner shaft 404 to a position proximal of the inner gripper 409. Prior to such movement the inner shaft 404 is disposed in a space between the inner gripper 409 to maintain the inner gripper 409 in the slots 120 to retain the ventricular member 112 in position on the delivery system 400. Movement along arrow A5 causes the inner shaft 404 to move out of position between the inner gripper 409. Such movement allows the inner gripper 409 to self-collapse to the configuration shown in FIG. 6F. This configuration provides a maximum outer dimension of the inner gripper 409 that is less than the inner diameter of the first hub 116 or in some embodiments moved out of the slots 120. In some configurations the inner gripper 409 do not fully deflect out of the slots 120 when the inner shaft 404 is withdrawn. In such configurations the inner gripper 409 may be sufficiently deflectable when the inner shaft 404 is withdrawn that the inner gripper 409 will be deflected by the heart valve prosthesis 100 as it moves off of the delivery system 400. For example the heart valve prosthesis 100 can be firmly engaged with two or more of the heart leaflets LF1, LF2, LF3 such that the leaflets retain the heart valve prosthesis 100 upon proximal movement of the guide handle 412 to withdraw the delivery system 400 from the heart.

FIG. 6G illustrates an additional embodiment of a delivery handle 414A. The features described with reference to FIG. 6G are optional. The delivery system 400 and in particular the delivery handle 414A can include one or more tubes or fluid sources 441 (e.g., tube 441a, tube 441b, tube 441c, tube 441d) configured to act as flushing sources for flushing catheter bodies of the system 400. The tubes 441a-d can allow for the delivery system 400 to be flushable with a fluid such as saline or any other biocompatible fluid. The fluid can flow through the tubes 441a-441d and delivery device and eventually into the blood and expelled. The tubes 441a-d when pressurized can assist in removing air from the delivery system 400, which can prevent the air from entering the bloodstream. Additionally, the tubes 441a-d when pressurized can prevent/remove blood from entering areas of the delivery device 400. The tubes 441a-441d when pressurized can prevent/reduce friction when moving the sliders 436, 440, 456, 460 to move different catheter bodies of the system 400.

The tubes 441a-d can each be in fluid communication with a different annular space within the delivery system 400 that can correspond to the slider to which each tube 441a-d is connected. For example, tube 441a can be connected to the first slider 436. The tube 441b can be connected to the second slider 440. The tube 441c can be connected to the third slider 456. The tube 441d can be connected to the fourth slider 460.

The first tube 441a can be fluidically connected to a first annular space that extends from first slider 436 toward or to the distal tip 410 of the delivery system 400. The second tube 441b can be fluidically connected to a second annular space that extends from the second slider 440 toward or to the distal tip 410 of the delivery system 400. The third tube 441c can be fluidically connected to a third annular space that extends from the third slider 456 toward or to the to the distal tip 410 of the delivery system 400. The fourth tube 441d can be fluidically connected to a fourth annular space that extends from the fourth slider 460 toward or to the distal tip 410 of the delivery system 400. The annular spaces can be separate from each other. The annular spaces can be embedded within each other. For example, the fourth annular space can enclose the third annular space. The third annular space can enclose the second annular space. The second annular space can enclose the first annular space. In another embodiments, the fourth annular space can extend through the third annular space. The fourth and the third annular spaces can then extend through the second annular space. The fourth, third, and second annular spaces can extend through the first annular space. One or more of the annular spaces can be disposed between an inner surface of the outer sheath 406 and the outer surface of the inner sheath 408. One or more of the annular spaces can be disposed between an outer surface of the inner shaft 404 and the inner surface of the inner sheath 408.

A syringe 442 can be used to push the fluid through the tubes 441a-d. In some embodiments, more than one syringe 442 can be used. For example, one or a separate syringe can be used for each tube 441a-d. Fluid can be selectively inserted one or more annular spaces at a time. Fluid can be inserted into more than one or all annular spaces simultaneously. The fluid can exit the syringe 442 and travel through a manifold 443. The manifold 443 can direct the fluid to the appropriate tube 441a-d and corresponding annular space. The fluid will then travel through the annular spaces of the delivery system and ultimately out the distal end. As described above, this can flush any air out of the delivery system, can prevent or remove blood from the system, and/or can reduce friction or can eliminate friction when moving the sliders 436, 440, 456, 460. When present the manifold 443 can be supplied with a continuous supply of fluid, e.g., a drip of saline from an elevated saline bag.

In some embodiments, each tube 441a-d can be connected to a flow regulator, to the manifold 443 and/or to an IV fluid source. The IV fluid source can be an IV bag fed by gravity, a pressurized bag, and/or a fluid pump. The flow regulator, manifold, and IV fluid source can be configured such that each tube 441a-d receives a fixed flow rate for flushing. This can be beneficial when all tubes 441a-d are connected together flushing and can allow for flow to be preferentially directed to the tube 441a-d with least resistance and bypass more restrictive tubes 441a-d.

In another embodiment, a single infusion supply tube can be provided, such as by causing one of the tubes 441a-441d to be in fluid communication with each interstitial space between adjacent layers of the system 400. This approach benefits from simplicity but may require a higher infusion pressure to counter resistance in certain interstitial spaces of the system 400.

FIGS. 8A-9B have been discussed above in connection with ventricular member 112A and the ventricular member 112B which are configured to plug a lumen of the first hub 116. The plug can be achieved during the technique illustrated in FIG. 6F. In particular, as the fourth slider 460 is withdrawn according to arrow A5 the inner shaft 404 is withdrawn from within the lumen of the first hub 116 of the ventricular member 112B. This allows the deflectable flow control member 150, e.g., the deflectable flaps 154, of the ventricular member 112A to extend out into the lumen of the first hub 116. As the fourth slider 460 is withdrawn according to arrow A5 the inner shaft 404 is withdrawn from within the lumen of the first hub 116 of the ventricular member 112B. This allows the compressible flow control member 162 of the first hub 116 of the ventricular member 112B to extend out into the lumen of the first hub 116. The control of flow in the ventricular member 112A is immediate when the deflectable flaps 154 contact each other. The ventricular member 112A can allow for re-crossing the lumen of the first hub 116. The ventricular member 112A can allow for re-crossing the lumen of the first hub 116 in one technique. The control of flow in the ventricular member 112B can be increasing over time as the compressible flow control member 162 absorbs blood which may become embedded in pores of the member 162.

FIG. 7A show engagement of the heart valve prosthesis 100 with heart tissue in one experiment. The heart valve prosthesis 100 is depicted from the atrial sides of the valve. The second hub 216 is seen disposed around the first hub 116, both of which are on the atrial side of the line of coaptation.

FIG. 7B shows the same heart valve prosthesis 100 engaged with heart tissue. FIG. 7B is a view of from the ventricular side of a valve to which the prosthesis 100 has been applied, with a chordae tendineae CT in the foreground. The second end 136 are shown engaged with the concavity 240. Leaflet tissue is trapped between the arms 124 and the frame array 220. In some applications one or more region of the heart valve prosthesis 100 may be disposed in the line of coaptation LOC, e.g., not engaged with either leaflet as seen at the 2 o'clock position. The atrial member 212 can be covered by the cover 223 as discussed above such that backflow can be reduced, minimized or prevented even for portions of the heart valve prosthesis 100 where leaflet tissue is not trapped between an arm 124 and concavity 240 or other portion of the frame array 220.

FIG. 7C shows that in some applications a portion spanning an angle of about 120 degrees of the circumference of the heart valve prosthesis 100 can be provided in which direct engagement is provided between the arms 124 and the concavities 240 of the frame array 220, e.g., no valve tissue is trapped in this spanning portion. In some embodiments, the arms 124 can align in the center of the concavities 240. In some embodiments, the arms 124 can be off-center of the concavities 240. This can be due to interaction with or interference by tissue or other interferences. The cover 223 can inhibit back flow over the spanning portion. In other applications a spanning of about 10 degrees, about 20 degrees, about 30 degrees, about 40 degrees, about 50 degrees, about 60 degrees, about 70 degrees, about 80 degrees, about 90 degrees, about 100 degrees, about 110 degrees, about 130 degrees, about 140 degrees, about 150 degrees, about 160 degrees, about 170 degrees, about 180 degrees, or over a range of degrees including any combination of the foregoing numbers as end points. In some applications more than one portion of the circumference of the heart valve prosthesis 100 can provide direct engagement between the ventricular member 112 and the atrial member 212 with portions of the heart valve prosthesis 100 therebetween engaging valve tissue.

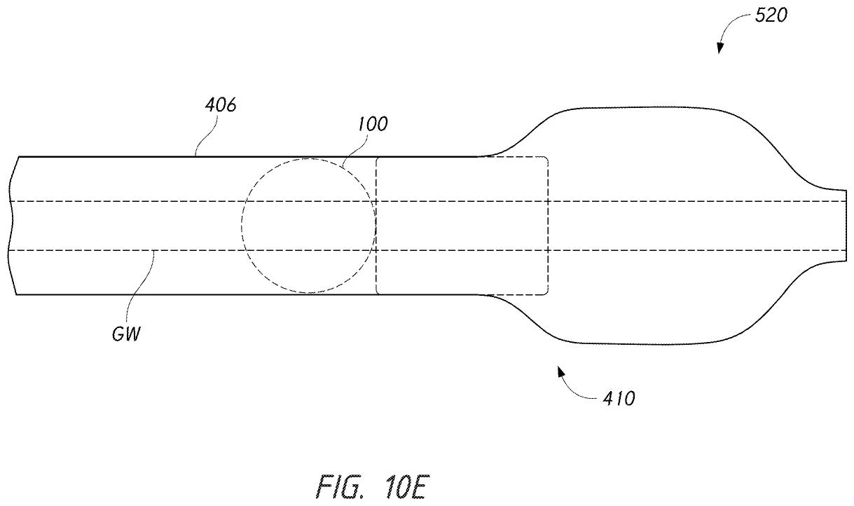

FIGS. 10A-10E illustrate exemplary nosecones to be coupled to or disposed at the distal tip 410 of the delivery system 400. The nosecones can allow the delivery system 400 to track over a guide wire GW within the vasculature. The nosecones can enable the heart valve prosthesis 100 to be pre-loaded at the distal end of the outer sheath 406. The nosecones can provide an atraumatic tip for the delivery system 400 so the delivery system 400 does not catch on or damage the vasculature or leaflets as the tip 410 traverses the line of coaptation of the valve leaflets. The nosecones can be provided in a location that blocks or otherwise protects the heart valve prosthesis 100 while the delivery system 400 is advanced and can be capable of transitioning to a second position or a second configuration that is out of the way of or at least less blocking of the heart valve prosthesis 100 such that the heart valve prosthesis 100 can pass through the distal tip 410 of the outer sheath 406.

FIGS. 10A-10B illustrate an example embodiment of a nosecone 500. FIG. 10A illustrates a nosecone 500. The nosecone 500 can be coupled to the distal tip 410 of the outer sheath 406. The nosecone 500 can be a distal portion or distal region of the distal tip 410. The nosecone 500 can have a first portion 501 that extends generally parallel to the outer sheath 406. The nosecone 500 can have a second portion 502 that extends at an angle relative to the outer sheath 406 toward a central axis of the sheath 406 to form a generally cone or other distally tapered shape. The second portion 502 can be configured to transition to an open state, as shown in FIG. 10B′.

The nosecone 500 can have a split tip. For example, the nosecone 500 can have one or more seams or splits 504 that allow the nosecone 500 to open away from a central axis. The nosecone 500 can have a plurality of seams or splits 504. For example, one, two, three, four or more seams or splits 504. The number of splits 504 can determine the number of sections 505 the nosecone 500 can be split into when in an open state. In some embodiments, the splits 504 can extend along both the first and second portions 501, 502 of the nosecone 500. In some embodiments, the splits 504 can extend along only the second portion 502 of the nosecone 500.

The splits 504 of the nosecone 500 can be secured together with a breakaway retention device or structure. For example, the breakaway retention device or structure can include a series of perforations, a zone provided with a weak adhesive, a zone with scoring along the splits 504 or any other suitable method. The breakaway retention device or structure can allow the nosecone 500 to be advanced in or as one piece and breakaway, split apart, or open once or as the heart valve prosthesis 100 is advanced through the nosecone 500. In some embodiments, the advancement of the heart valve prosthesis 100 can cause the nosecone 500 to separate at the splits 504.

FIGS. 10B and 10B′ illustrate the nosecone 500 transitioning from a closed state, position or configuration to an open state, position or configuration. In the closed state, the delivery system 400 is advanced to the delivery location. The nosecone 500 is closed to prevent the heart valve prosthesis 100 from exiting the delivery system 400. In the open state, the delivery system 400 is at the delivery location and the nosecone transitions to an open state to allow for delivery of the heart valve prosthesis 100.

The nosecone 500 can have an interlock 508. The interlock 508 can be configured to retain the nosecone 500 in a closed state during advancement of the delivery system 400. By pushing the distal tip 410 of the delivery system 400 into the interlock 508, the nosecone 500 can be held in a closed position. The nosecone 500 can be opened by releasing the interlock 508 by withdrawing the distal tip 410 and pushing the heart valve prosthesis 100 through the nosecone 500. The nosecone 500 can the open away from the central axis, as designated by arrow 2.

FIGS. 10C and 10C′ illustrate another example embodiment of a nosecone 512. FIG. 10C illustrates the nosecone 512 in a closed state or configuration and FIG. 10C′ illustrates the nosecone 512 in an open state or configuration. The nosecone 512 can be coupled to the distal tip 410 of the outer sheath 406. The nosecone 512 can comprise a flexible material. The flexible material can be configured to open or expand as the heart valve prosthesis 100 passes through the nosecone 512. After the heart valve prosthesis 100 is delivered the nosecone 512 can revert to its original closed shape. FIG. 10C′ shows elastic expansion of the nosecone 512, though some deformation is acceptable given that the function of the nosecone is no longer needed after the heart valve prosthesis 100 has been deployed.

FIGS. 10D and 10D′ illustrate another example embodiment of a nosecone 516. FIG. 10D illustrates the nosecone 516 in a closed state or configuration and FIG. 10D′ illustrates the nosecone 512 in an open state or configuration. The nosecone 516 can be configured as an additional outer sheath disposed around outer sheath 406. The nosecone 516 can have a tip portion and an outer sheath portion. The tip portion and outer sheath portion can enclose the outer sheath 406. The nosecone 516 can slide over the outer sheath 406 to expose the distal tip 410 of the outer sheath 406. The nosecone 516 can be withdrawn or pulled back by pulling and stretching the nosecone 516 over the outer sheath 406, as designated by the arrows. In one technique the outer sheath 406 can be advanced out of the nosecone 516. In another technique a simultaneous motion of the nosecone 516 and the outer sheath 406 can be provided to move the tip of the sheath 406 out of the nosecone 516. The heart valve prosthesis 100 can be delivered through the exposed distal tip 410 of the outer sheath 406.

FIG. 10E illustrates another example embodiment of a nosecone 520. The nosecone 520 can be an inflatable balloon tip that is disposed inside or partially coupled to an outer surface of the distal tip 410 of the outer sheath 406. The inflatable balloon tip nosecone 520 can be inflated by a lumen located inside the distal tip 410 to prevent the heart valve prosthesis 100 from exiting the outer sheath 406. The inflated tip can block the passage inside the distal tip 410 to block egress of the prosthesis 100. The inflatable tip can have a hemispherical, tapered, or conical profile that can function as a nosecone until delivery of the heart valve prosthesis 100. In some embodiments, the heart valve prosthesis 100 can be advanced along the guidewire GW and the inflatable balloon tip can surround the guidewire and be inflated to prevent the heart valve prosthesis 100 from exiting the delivery system 400. The inflatable balloon tip nosecone 520 can then be deflated and drawn back past the heart valve prosthesis 100 such that the heart valve prosthesis 100 can exit the outer sheath 406. Or, the prosthesis 100 can be advanced over the inflatable balloon tip nosecone 520 when the nosecone 520 is deflated.

Among the advantages of the prostheses, delivery systems, nosecones, and methods disclosed herein is the fact that the procedure can be used to deploy the prosthesis without anticoagulation therapy.

### Terminology

As used herein, the relative terms “proximal” and “distal” can be defined from the perspective of the implant. Thus, proximal refers to the direction of the portion of the implant to be disposed in the right atrium and distal refers to the portion of the implant to be disposed in the right ventricle.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments.

The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 1” includes “1.” Phrases preceded by a term such as “substantially,” “generally,” and the like include the recited phrase and should be interpreted based on the circumstances (e.g., as much as reasonably possible under the circumstances). For example, “substantially spherical” includes “spherical.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C; A and B; A and C; B and C; and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y and at least one of Z to each be present.

Although certain embodiments and examples have been described herein, it should be emphasized that many variations and modifications may be made to the heart valve prostheses and delivery systems shown and described in the present disclosure, the elements of which are to be understood as being differently combined and/or modified to form still further embodiments or acceptable examples. All such modifications and variations are intended to be included herein within the scope of this disclosure. A wide variety of designs and approaches are possible. No feature, structure, or step disclosed herein is essential or indispensable.

Some embodiments have been described in connection with the accompanying drawings. However, it should be understood that the figures are not drawn to scale. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Moreover, while illustrative embodiments have been described herein, it will be understood by those skilled in the art that the scope of the inventions extends beyond the specifically disclosed embodiments to any and all embodiments having equivalent elements, modifications, omissions, combinations or sub-combinations of the specific features and aspects of the embodiments (e.g., of aspects across various embodiments), adaptations and/or alterations, and uses of the inventions as would be appreciated by those in the art based on the present disclosure. The limitations in the claims are to be interpreted broadly based on the language employed in the claims and not limited to the examples described in the present specification or during the prosecution of the application, which examples are to be construed as non-exclusive. Further, the actions of the disclosed processes and methods may be modified in any manner, including by reordering actions and/or inserting additional actions and/or deleting actions. It is intended, therefore, that the specification and examples be considered as illustrative only, with a true scope and spirit being indicated by the claims and their full scope of equivalents.

Any methods disclosed herein need not be performed in the order recited. The methods disclosed herein include certain actions taken by a practitioner; however, they can also include any third-party instruction of those actions, either expressly or by implication. For example, actions such as “inserting a delivery catheter into a right internal jugular vein” include “instructing insertion of a delivery catheter into a right internal jugular vein.”

1. A heart valve prosthesis, comprising:
a ventricular member configured to be advanced into a ventricle of a heart, the ventricular member comprising a first hub comprising slots, the first hub configured to be disposed adjacent to a line of coaptation of a first heart leaflet and a second heart leaflet and an array of arms, each arm having a first end connected to the first hub and a second end opposite the first end, the second end of each arm deflected towards the first hub in a free state, the second ends of adjacent arms not being connected to each other, one or more of the second ends configured to be placed into direct contact with the first heart leaflet and one or more of the second ends configured to be placed into direct contact with the second heart leaflet; and
an atrial member configured to be advanced into an atrium adjacent to the line of coaptation of the first heart leaflet and the second heart leaflet, the atrial member comprising a second hub comprising tangs and an array of petals, the tangs configured for locking into the slots of the first hub to engage the second hub with the first hub when the atrial member and the ventricular member are assembled, each petal of the array of petals having a base end connected to the second hub and an outer end opposite the base end, the outer end of each petal deflected away from the second hub, adjacent petals bounded by a shared inner strut and a separate outer strut forming a concavity at a junction of the shared inner strut and the separate outer strut;
wherein the atrial member is separate from and moveable relative to the ventricular member prior to the first hub and the second hub being engaged; and
wherein in an engaged configuration the arms of the ventricular member are pressed into the concavity of the atrial member.

2. The heart valve prosthesis of claim 1, wherein the ventricular member and the atrial member are made of a shape-memory alloy.

3. The heart valve prosthesis of claim 1, wherein the ventricular member and the atrial member comprise Nitinol.

4. The heart valve prosthesis of claim 1, wherein the second end of each arm has a tip, the tip having a narrowed portion and an enlarged portion, the enlarged portion distal to the narrowed portion.

5. The heart valve prosthesis of claim 1, wherein a ventricular clamp diameter measured to a circle scribed by the second ends of the arms, is less than an atrial clamp diameter measured to a circle scribed by the outer ends of the petals.

6. The heart valve prosthesis of claim 1, wherein a ventricular clamp diameter measured to a circle scribed by the second ends of the arms is about 20 mm.

7. The heart valve prosthesis of claim 1, wherein an atrial clamp diameter measured to a circle scribed by the outer ends of the petals is about 25 mm.

8.-27. (canceled)

28. A system for delivering a heart valve prosthesis, comprising:
a delivery device comprising:
a delivery handle, comprising:
a housing; and
a plurality of sliders disposed in the housing;
a plurality of removable lockouts configured to prevent motion of the plurality of sliders in a first state and to allow a range of motion in a second state;
a sheath assembly comprising:
an outer sheath; and
an inner sheath disposed within the outer sheath, the outer sheath moveable relative to the inner sheath; and
a guide handle configured to retract the outer sheath; and
a prosthesis comprising:
a ventricular member comprising at least three arms, each arm having a first end coupled to a hub; and
an atrial member comprising a hub configured to couple with the hub of the ventricular member and at least one centering feature comprising a concavity for centering at least one of the at least three arms of the ventricular member;
wherein the delivery handle is configured to control movement and positioning of the prosthesis within a heart by movement of the plurality of sliders and plurality of removable lockouts.

29. The system of claim 28, further comprising a nosecone coupled to a distal end of the outer sheath.

30. The system of claim 29, wherein the nosecone comprises a plurality of slits configured to open the nosecone to allow the prosthesis to exit the outer sheath.

31. The system of claim 29, wherein the nosecone comprises a flexible tip configured to expand to allow the prosthesis to exit the outer sheath.

32. The system of claim 29, wherein the nosecone surrounds the outer sheath and is configured to be retracted to allow the prosthesis to exit the outer sheath.

33. The system of claim 28, further comprising an inflatable balloon disposed within the outer sheath, the inflatable balloon configured to be inflated to prevent the prosthesis from exiting the outer sheath and configured to be deflated to allow the prosthesis to exit the outer sheath.

34. The system of claim 28, further comprising a source of fluid coupled with the housing and configured to supply a fluid to a space disposed between an inner surface of the outer sheath and an outer surface of the inner sheath.

35. The system of claim 34, further comprising a source of fluid coupled with the housing and configured to supply a fluid to a plurality of spaces disposed between an inner surface of the outer sheath and an outer surface of the inner sheath.

36. The system of claim 34, further comprising a plurality of fluid sources coupled with a plurality of spaces disposed between an inner surface of the outer sheath and an outer surface of the inner sheath, one of the fluid sources of the plurality of fluid sources supplying fluid to each one of the plurality of spaces between the inner surface of the outer sheath and the outer surface of the inner sheath.

37. A method of performing a procedure in a heart, comprising:
advancing a delivery catheter to the heart;
passing a ventricular member through the delivery catheter and into a ventricle of the heart, the ventricular member comprising at least three arms, each arm having a first end coupled to a hub;
passing an atrial member through the delivery catheter and into an atrium of the heart, the atrial member comprising at least one centering feature comprising a concavity for centering at least one of the at least three arms of the ventricular member;
aligning the ventricular member and the atrial member such that tissue is compressed between the ventricular member and the atrial member;
inserting the hub of the ventricular member to a hub of the atrial member; and
securing the ventricular member to the atrial member.

38. The method of claim 37, wherein the delivery catheter is coupled to a delivery handle comprising:
a plurality of sliders disposed in a housing; and
a plurality of removable lockouts configured to prevent motion of the plurality of sliders in a first state and to allow a range of motion in a second state;
wherein the plurality of sliders and the plurality of removable lockouts are configured to pass the ventricular member and the atrial member through the delivery catheter.

39. The method of claim 37, wherein the tissue is a tricuspid valve.

40. The method of claim 37, further comprising supplying a fluid to a space disposed within the delivery catheter via a source of fluid coupled to a delivery handle coupled to the delivery catheter.

41. The method of claim 40, further comprising suppling a fluid to a plurality of spaces disposed within the delivery catheter.

42. The method of claim 37, further comprising opening a plurality of slits in a nosecone coupled to a distal end of the delivery catheter, wherein opening the plurality of slits allows the ventricular member and the atrial member to pass through the distal end of the delivery catheter.

43.-49. (canceled)
